(19) United States
(12) Patent Application Publication
(10) Pub. No.: US 2008/0118437 A1
(43) Pub. Date: May 22, 2008

(54) Diagnosis and treatment O prostate cancer

(75) Inventors: Kenneth Pienta, Ann Arbor, MI (US); Robert D. Loberg, Saline, MI (US)

Correspondence Address: MEDLEN & CARROLL, LLP, 101 HOWARD STREET, SUITE 350, SAN FRANCISCO, CA, 94105, US

(73) Assignee: Regents of the University of Michigan, Ann Arbor, MI (US)

(21) Appl. No.: 11/712,753

(22) Filed: Mar. 1, 2007

Related U.S. Application Data
(60) Provisional application No. 60/777,938, filed on Mar. 1, 2006.

(51) Int. Cl. A61K 39/395; A61K 48/00; C12Q 1/68; A61K 38/00; G01N 33/53; A61K 49/00

(52) U.S. Cl. 424/9.2; 424/138.1; 514/44; 435/6; 514/2; 435/7.1

(57) ABSTRACT

The present invention relates to compositions and methods for cancer diagnosis, treatment and research, including but not limited to, cancer markers and uses of cancer markers. In particular, the present invention provides compositions and methods for targeting MCP-1 in prostate cancer.

This application claims priority to provisional patent application Ser. No. 60/777,938, filed Mar. 1, 2006, which is herein incorporated by reference in its entirety.

This Application was supported in part by NIH grant 1 PO1 CA093900-02. The government may have certain rights in the invention.

### FIELD OF THE INVENTION

The present invention relates to compositions and methods for cancer diagnosis, treatment and research, including but not limited to, cancer markers and uses of cancer markers. In particular, the present invention provides compositions and methods for targeting MCP-1 in prostate cancer.

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 184,500 American men will be diagnosed with prostate cancer and 39,200 will die in 2001.

Prostate cancer is typically diagnosed with a digital rectal exam and/or prostate specific antigen (PSA) screening. An elevated serum PSA level can indicate the presence of PCA. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen (PSA) screening has led to earlier detection of PCA and significantly reduced PCA-associated fatalities. However, the impact of PSA screening on cancer-specific mortality is still unknown pending the results of prospective randomized screening studies (Etzioni et al., J. Natl. Cancer Inst., 91:1033 [1999]; Maattanen et al., Br. J. Cancer 79:1210 [1999]; Schroder et al., J. Natl. Cancer Inst., 90:1817 [1998]). A major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of additional serum and tissue biomarkers to supplement PSA screening is needed. Additional therapies are also needed, especially for advanced disease.

### SUMMARY OF THE INVENTION

The present invention relates to compositions and methods for cancer diagnosis, treatment and research, including but not limited to, cancer markers and uses of cancer markers. In particular, the present invention provides compositions and methods for targeting MCP-1 in prostate cancer.

Accordingly, in some embodiments, the present invention provides methods and compositions for treating prostate cancer and/or preventing metastasis of prostate cancer by decreasing MCP-1 activity. The present invention further provides diagnostic methods for diagnosing prostate cancer and for identifying prostate cancer that is likely to or at an increased risk to metastasize. The present invention additionally provides research applications (e.g., drug screening applications).

For example, in some embodiments, the present invention provides a method of inhibiting the growth of a cancer cell, comprising contacting the cancer cell with an agent that inhibits an activity of MCP-1. In some embodiments, the cancer cell is a prostate cancer cell (e.g., a metastatic prostate cancer cell). In some embodiments, the metastatic prostate cancer cell is a bone metastasis. In some embodiments, the agent is a small molecule that inhibits a biological activity of MCP-1. In other embodiments, the agent is an antibody that binds to the MCP-1. In yet other embodiments, the agent is an antisense or siRNA that inhibits the expression of the MCP-1. In still further embodiments, the agent is an MCP-1 TRAP. In certain embodiments, the agent is a combination of one or more of the described agents. For example, in some embodiments, the agent is a combination of an antibody that binds to MCP-1 and a known chemotherapy agent (e.g., TAXOTERE (docetaxel)). In some embodiments, the combination of an antibody that binds to MCP-1 and a known chemotherapy agent is administered first, followed by administration of only the antibody. In some embodiments, the administration of the antibody is continued for a maintenance period. In some embodiments, the cancer cell is in an organism (e.g., a human or a non-human mammal).

The present invention further provides a method of preventing metastasis (e.g., to the bone) of a cancer cell, comprising contacting the cancer cell with an agent that inhibits an activity of MCP-1.

In other embodiments, the present invention provides a composition comprising an agent that inhibits a biological activity of MCP-1. In some embodiments, the composition further comprises a pharmaceutically acceptable carrier. In some embodiments, the agent is a small molecule that inhibits a biological activity of MCP-1, an antibody that binds to the MCP-1, a known chemotherapy agent, an antisense or siRNA that inhibits the expression of the MCP-1, or a combination of one or more of the agents.

The present invention additionally provides a method of characterizing a prostate tissue sample, comprising measuring the level of expression of MCP-1 in the tissue. In some embodiments, an increase in MCP-1 relative to the level in a non-cancerous prostate tissue is indicative of the presence of prostate cancer in the tissue. In other embodiments, an increase in MCP-1 relative to the level in a non-cancerous prostate tissue is indicative of the presence of metastatic prostate cancer in the tissue. In still further embodiments, an increase in MCP-1 relative to the level in a non-cancerous prostate tissue is indicative of the presence of prostate cancer in the tissue that is likely to metastasize. In some embodiments, the prostate tissue sample is a biopsy sample.

In yet other embodiments, the present invention provides a method of screening compounds, comprising contacting a cell expression MCP-1 with a test compound; and measuring a biological activity of MCP-1 in the presence of the test compound relative to the level in the absence of the test compound. In some embodiments, the cell is a prostate cancer cell. In certain embodiments, the prostate cancer cell is in an organism (e.g., a non-human mammal). In some embodiments, the biological activity is promotion of metastasis of the prostate cancer cell. In other embodiments, the biological activity is expression of the MCP-1. In some embodiments, the test compound is a small molecule, an antibody, an siRNA, an MCP-1 TRAP, an antisense nucleic acid, or a combination of one or more of the described agents. In preferred embodiments, the test compound inhibits or decreases the biological activity of MCP-1.

### DESCRIPTION OF THE FIGURES

FIG. 1 shows representative cytokine antibody arrays comparing normal versus tumor microenvironments. Arrays demonstrate upregulation of MCP-1 (boxed in area, replicates of two) in the normal bone (A) compared to the bone tumor (B) microenvironments. C) Graphical representation of MCP-1 expression by cytokine antibody array analysis in normal adrenal, adrenal tumor, normal liver, liver tumor, normal bone, and bone tumor. Data is represented as mean ±SD, * indicates p value <0.001.

FIG. 2 shows enzyme-linked immunoabsorbant assay of MCP-1 release. A) Two prostate cancer cell lines (PC-3 and VCaP), bone marrow endothelial cells (HBME), osteoblasts (OB), adipocytes (NIH-3T3 L1) cells were plated in 6 well plates. B) Aortic endothelial cells (HAEC), microvascular endothelial cells (HDMVEC), and bone marrow endothelial cells (HBME) were plated in 6 well plates and conditioned media was collected after 24 hours. MCP-1 concentrations are reported as [pg/mL] per 100,000 cells and the data is presented as mean ±SD, * indicates p value <0.001. C) HBME cells were plated in the lower chamber of a modified Boyden Chamber and allowed to condition media for 24 hours. Data is presented as mean ±SD, * indicates p value <0.01.

FIG. 3 shows that MCP-1 is a chemoattractant for prostate cancer cells and stimulates cell migration. A) Prostate cancer cell migration in response to MCP-1 was assessed by using recombinant human MCP-1 (1-100 ng/mL) in A) PC-3 and C) VCaP cells. B) PC-3 and D) VCaP cell dose dependent migration in rhMCP-1 was attenutated by neutralizing anti-MCP-1 antibodies (2 mg/mL) and the anti-mouse MCP-1/JE antibody (2 mg/mL).

FIG. 4 shows that MCP-1 induces Akt phosphorylation in prostate cancer cells. PC-3 (A) and VCaP (C) were treated with MCP-1 (100 ng/mL) for 0 to 30 min and phosphorlation of Akt was determined by Western Blot analysis. PC-3 (B) and VCaP (D) were treated with increasing concentrations of MCP-1 (0-100 ng/mL) for 30 min and phosphorlation of Akt was determined. E) GSK3α/β and F) p70 S6 kinase, two downstream targets of Akt, were analyzed in PC-3 cells stimulated with MCP-1 (100 ng/mL).

FIG. 5 shows that MCP-1 induces PC-3 and VCaP cell proliferation via a PI3K/Akt dependent mechanism. PC-3 (A) and VCaP (B) cells were plated in a 96 well plate and stimulated with increasing concentrations of rhMCP-1 (1-100 ng/mL) for 24 to 96 hours (solid lines, open symbols). LY294002 (PI3K inhibitor) was added at 1 μM (dashed lines, solid symbols) and compared to the vehicle treated control (solid line, solid symbol).

FIG. 6 shows that MCP-1 induces actin rearrangement and lamellipodial formation in PC-3 cells. PC-3 cells were stimulated with MCP-1 (100 ng/mL) (b) in the presence of a neutralizing MCP-1 antibody (4 μg/mL) (c), a CCR2b inhibitor (1 μM) (d) or SDF-1 (200 ng/mL) (e) as a positive control. The nucleus was stained with DAPI and actin was visualized with Rhodamine Phallodin.

FIG. 7 shows in vivo bioluminescent imaging of PC-3^{Luc} cell metastasis during MCP-1 systemic targeted therapy. A) Mice received PC-3^{Luc} cells by intracardiac injection. Beginning on Day 14 mice received anti-human IgG (♦), anti-mouse cVaM (◯), anti-human MCP-1 (), or anti-mouse MCP-1/JE [symbol] at 2 mg/Kg twice weekly by intraperitoneal injection. B) anti-human IgG, C) anti-human MCP-1, D) anti-mouse cVaM, and E) anti-mouse MCP-1/JE.

FIG. 8 shows Table 1.

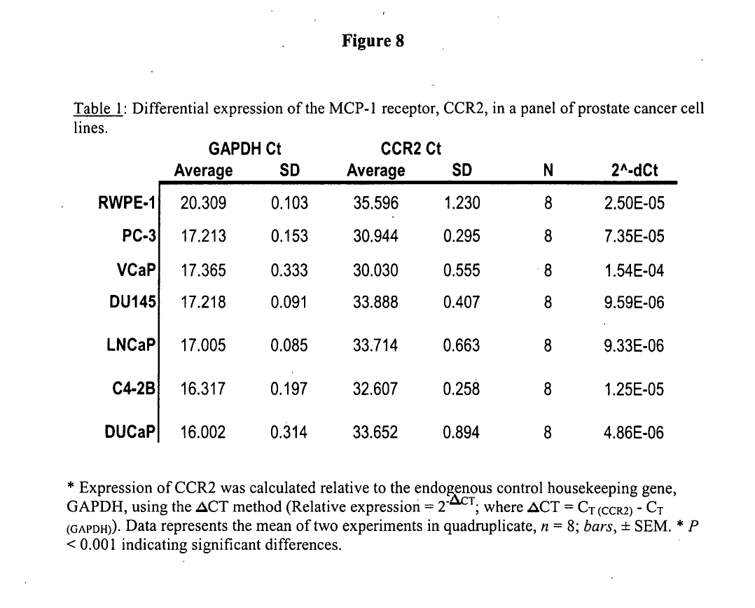

FIG. 9 shows exemplary MCP-1 TRAP molecules.

FIG. 10 shows that CNTO888 inhibits PC-3Luc cell proliferation and migration in vitro. A) PC-3 cells were stimulated with CCL2 [10 ng/mL (▪) or 100 ng/mL (▪)] for 72 hours in the presence or absence of CNTO888 (30 μg/mL), C1142 (30 μg/mL), or CNTO888+C1142 (30 μg/mL each). B) PC-3 cell migration was measured in response to hrCCL2 (100 ng/mL). C) Immunoblot analysis of Akt, p70S6 kinase, and MAPK p44/p42 activation. 1-control, 2-CCL2 (100 ng/mL, 24 hours), 3-CCL2 (100 ng/mL)+C1142 (30 μg/mL), 4-CCL2 (100 ng/mL)+CNTO888 (30 μg/mL), 5-C1142 (30 μg/mL), 6-CNTO888 (30 μg/mL).

FIG. 11 shows that CCR2 expression correlates with prostate cancer progression and metastasis. CCR2 expression was analyzed by tissue microarray analysis (TMA) and demonstrated epithelial cell staining in normal (A,B), primary prostate cancer (C,D), and metastatic prostate cancer (E,F). Graphical analysis of TMAs showed a significant increase in CCR2 expression (G) and correlated with disease progression and Gleason Score (H). No difference was seen in CCR2 expression between soft tissue metastases and bone metastases (I).

FIG. 12 shows in vivo bioluminescent imaging of PC-3 Luc cell metastasis during systemic CCL2-targeted therapy. A) Mice received PC-3Luc cells by intracardiac injection. Beginning on Day 14 mice received huIgG control antibody (♦), mouse antibody control C1322 (◯), anti-human CCL2 CNTO888 (∩), or anti-CCL2/JE C1142 [symbol] at 2 mg/Kg twice weekly by i.p. injection. B) Tumor burden was calculated as a percent control antibodies on Day 35 for comparison and graphed as mean percentage ±standard deviation (* p<0.01). C-F) Pictures illustrate images captured on Day 35 from representative animals from each group: C) huIgG, D) CNTO888, E) C1322, and F) C1142.

FIG. 13 shows that anti-CCL2 antibodies decrease bone-specific tumor burden in vivo. A) Tumor burden in the tibia was analyzed independently and tibia-specific tumor burden was assessed over the five week treatment period. B) Final day (Day 35) tibia-specific tumor burden demonstrated significant reduction by CCL2 inhibition, by either anti-tumor or anti-host CCL2 antibodies (* p<0.001). C) The number of metastases was identified by gross examination of luciferase signal for each animal and graphed as the total number of soft tissue (ST) versus bone (B) metastases (ns—not significant, * p<0.05).

FIG. 14 shows the efficacy of single agent anti-CCL2 antibodies compared to single agent docetaxel in vivo. A) Inhibition of CCL2 was compared to docetaxel (MTD—40 mg/Kg). Beginning on Day 14, mice received CNTO888 (∩), C1142 (□), CNTO888+C1142 [symbol] or docetaxel (⋄) by intraperitoneal injection. B) Total tumor burden at Day 35.

FIG. 15 shows that Anti-CCL2 antibodies in combination with docetaxel induce tumor regression in vivo. Beginning on Day 14, mice received docetaxel+CNTO888 (∩), docetaxel+C1142 (□), docetaxel+CNTO888+C1142 [symbol] or docetaxel (⋄) by i.p. injection.

FIG. 16 shows that HBME cells cultured in either PC-3 or VCaP conditioned media secreted significantly higher levels of CCL2 compared to HAEC or HDMVEC cultured in similar conditioned media.

FIG. 17 shows that stimulation of HBME, HAEC and HDVMEC cells with PTHrP (10 nM) increased their respective synthesis of CCL2.

FIG. 18 shows that primary mouse calvaria osteoblasts increased CCL2 expression when stimulated with PTHrP.

FIG. 19 shows that the presence of PC-3 and VcaP cell xenografts significantly increased the levels of CCL2 expression in the bone marrow compared to control animals.

FIG. 20 shows that overall tumor burden was significantly decreased in tibias of mice receiving C1142 (anti-CCL2/JE) antibodies compared to control antibodies (C1322).

FIG. 21 shows a decrease in TRAP5b serum concentration in animals receiving anti-CCL2 antibodies compared to controls.

FIG. 22 shows attenuation of tumor growth by inhibition of CCL2. Tumor volume was monitored weekly by caliper measurement in mice receiving C1142 versus C1322 (A) and huIgG versus CNTO888 (B). Immunohistochemical analysis of the xenograft tumors displayed normal prostate adenocarcinoma morphology by H&E in mice treated with C1322 (C), C1142 (D), huIgG (G), or CNTO 888 (H). Neovascularization was visualized by immunohistochemical staining of CD31 (E,F,I,J). K) The ability of CCL2 inhibition to reduce blood vessel formation was analyzed using an in vitro tube formation assay.

FIG. 23 shows that inhibition of CCL2 reduced the number of infiltrating macrophages in the VCaP xenograft. Macrophage infiltration was visualized by immunohistochemical staining for CD68 (A-D). E) The number of infiltrating macrophages is shown.

FIG. 24 shows that inhibition of tumor-derived CCL2 reduced the number of migrating U937 monocytes.

FIG. 25 shows that inhibition of CCL2 reduces the amount of proliferation in the VCaP xenografts. Apoptosis was visualized by immunohistochemical staining with ApopTag (A-D, H-K). Proliferation was visualized by immunohistochemical staining for Ki67 (E-F, L-M).

FIG. 26 show that inhibition of CCL2 attenuates Akt and MAPK p44/p42 activity. Tumor specimens were collected and stained with anti-phospho Akt (A-B, E-F) or anti-phospho p44/p42 (C-D, G-H) to visualize intratumoral activity of these signaling pathways. I) Immunoblot analysis of VCaP cells stimulated with CCL2 (100 ng/mL for 24 hours) in the presence of anti-CCL2 antibodies.

FIG. 27 shows that CCL2 stimulates Akt and p70S6 kinase activation in VCaP cells. CCL2 induces Akt phosphorylation (A) and p70S6 kinase phosphorylation (B) in a dose dependent fashion.

### DEFINITIONS

To facilitate an understanding of the present invention, a number of terms and phrases are defined below:

The terms “MCP-1” and “CCL2” are used interchangeably to refer to CC chemokine ligand 2 or monocyte chemoattractant protein 1. The CCL2 gene maps to 2 chromosome 17q11.2-q12 and comprises a 99 amino acid precursor protein that when processed and secreted is 75 amino acids in size. CCL2 mRNA is described by Genbank ID NO: NM_{—}002982.

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxygenin; luminogenic, phosphorescent or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “biological activity of MCP-1” refers to any activity normally associated with MCP-1 in a cell or in vitro. Biological activities of MCP-1 include both activities associated with the protein in normal cells, as well as activities associated with MCP-1 in cancer cells or cancerous tissue. Biological activities of MCP-1 include, but are not limited to, expression of MCP-1 mRNA or protein, promotion of cancer cell proliferation, and promotion of cancer metastasis.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis (e.g., a CT scan showing a mass or increased PSA level) but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes (e.g., MCP-1).

As used herein, the term “characterizing prostate tissue in a subject” refers to the identification of one or more properties of a prostate tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized by the identification of the expression of MCP-1.

As used herein, the term “prostate cancer tissue sample” refers to a sample consisting substantially (e.g., greater than 80%, preferably greater than 90%, and even more preferably greater than 99%) of prostate cells (e.g., that have been classified as cancerous by a pathologist or other qualified individual or instrument). Generally, the prostate is removed from a subject by surgery (e.g., radical prostatectomy) and a section of the prostate suspected of comprising cancerous cells is analyzed.

As used herein, the term “cancer marker genes” refers to a gene whose expression level, alone or in combination with other genes, is correlated with the presence of cancer or prognosis of cancer. The correlation may relate to either an increased or decreased expression of the gene. For example, the expression of the gene may be indicative of cancer, or lack of expression of the gene may be correlated with poor prognosis in a cancer patient. Cancer marker expression may be characterized using any suitable method, including but not limited to, those described herein.

As used herein, the term “a reagent that specifically detects expression levels” refers to reagents used to detect the expression of one or more genes (e.g., MCP-1). Examples of suitable reagents include but are not limited to, nucleic acid probes capable of specifically hybridizing to the gene of interest, PCR primers capable of specifically amplifying the gene of interest, and antibodies capable of specifically binding to proteins expressed by the gene of interest. Other non-limiting examples can be found in the description and examples below.

As used herein, the term “detecting a decreased or increased expression relative to non-cancerous prostate control” refers to measuring the level of expression of a gene (e.g., the level of mRNA or protein) relative to the level in a non-cancerous prostate control sample. Gene expression can be measured using any suitable method, including but not limited to, those described herein.

As used herein, the term “detecting a change in gene expression (e.g., MCP-1) in said prostate cell sample in the presence of said test compound relative to the absence of said test compound” refers to measuring an altered level of expression (e.g., increased or decreased) in the presence of a test compound relative to the absence of the test compound. Gene expression can be measured using any suitable method.

As used herein, the term “instructions for using said kit for detecting cancer in said subject” includes instructions for using the reagents contained in the kit for the detection and characterization of cancer in a sample from a subject. In some embodiments, the instructions further comprise the statement of intended use required by the U.S. Food and Drug Administration (FDA) in labeling in vitro diagnostic products.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “initial diagnosis” refers to results of initial cancer diagnosis (e.g. the presence or absence of cancerous cells). An initial diagnosis does not include information about the stage of the cancer of the risk of prostate specific antigen failure.

As used herein, the term “biopsy tissue” refers to a sample of tissue (e.g., prostate tissue) that is removed from a subject for the purpose of determining if the sample contains cancerous tissue. In some embodiment, biopsy tissue is obtained because a subject is suspected of having cancer. The biopsy tissue is then examined (e.g., by microscopy) for the presence or absence of cancer.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to compositions and methods for cancer diagnosis, treatment and research, including but not limited to, cancer markers and uses of cancer markers. In particular, the present invention provides compositions and methods for targeting MCP-1 (CCL2) in prostate cancer.

The predominance of prostate cancer metastasis to bone has been well documented from three independent autopsy series and has been reported to occur with 85% frequency in patients with advanced hormone refractory prostate cancer (Shah et al., (2004) Cancer Res 64, 9209-9216). Metastasis is a process that is defined by a series of sequential steps resulting in end organ tumor metastasis via a migratory pattern that appears to be both directed, specific and predictable (Shah et al., supra). The present invention is not limited to a particular mechanism. Indeed, an understanding of the mechanism is not necessary to practice the present invention. Nonetheless, one mechanism that has been proposed to explain the enhanced frequency of bone metastases in prostate cancer is the preferential adhesion to the bone marrow endothelium (Cooper and Pienta, K. J. (2000) Prostate Cancer Prostatic Dis 3, 6-12). However, simply adhering to the endothelial wall is not sufficient to invade an organ, thus, some cancer cells must acquire the ability to migrate from the luminal side of the endothelial cells into the surrounding tissue in response to chemotactic molecules released by stromal cells. Several chemokines, including stromal cell-derived factor 1 (SDF-1/CXCL12) and recently MCP-1 (CCL2) (Taichman et al., (2002) Cancer Res 62, 1832-1837; Vanderkerken et al., (2002) Clin Exp Metastasis 19, 87-90; Kulbe et al., (2004) Int J Dev Biol 48, 489-496) have been shown to promote chemotactic migration of cancer cells (prostate for SDF1 (Taichman et al., supra); myeloma for MCP-1 (Vanderkerken et al., supra)). MCP-1 is a member of the CC β chemokine family and was originally known to promote monocyte and macrophage migration to sites of inflammation (Ohta et al., (2003) Int J Oncol 22, 773-778; Balkwill, (2003) Semin Immunol 15, 49-55).

MCP-1 has previously been shown to be an important determinant of the macrophage and monocyte infiltration in breast, cervix and pancreatic carcinomas (Balkwill and Mantovani, (2001) Lancet 357, 539-545). Recent studies have demonstrated that MCP-1 localizes to tumor epithelial cells (Negus et al., (1995) J Clin Invest 95, 2391-2396). The levels of MCP-1 expression has been correlated with the involvement of lymphocytes and macrophages localization in secondary sites of tumor formation (Negus et al., (1997) Am J Pathol 150, 1723-1734). There is growing evidence to suggest that MCP-1 may act directly on the epithelial cells of several human carcinomas and may regulate the migration and invasive properties of the tumor cells resulting in an enhanced metastatic potential. Youngs et al (1997) demonstrated a dose-dependent migratory response of breast cancer cells to increasing concentration of exogenous MCP-1 (Youngs et al., (1997) Int J Cancer 71, 257-266). Additionally, MCP-1 expression has been shown to correlate with progression in pancreatic cancer (Neumark et al., (2003) Int J Cancer 106, 879-886) and breast cancer (Saji et al., (2001) Cancer 92, 1085-1091).

Recognition of prostate cancer metastasis to bone as a lethal phenotype is leading to the design of new therapies directed at both the cancer cell as well as the bone microenvironment. Tumor cells in the bone interact with the extracellular matrix (ECM), stromal cells, osteoblasts, osteoclasts, and endothelial cells to coordinate a sophisticated series of interactions to promote tumor cell survival and proliferation leading to morbidity and mortality for patients with advanced prostate cancer (Logothetis et al., (2005) Nat Rev Cancer 5, 21-28; Pienta et al., (2005) Clin Prostate Cancer 4, 24-30). There is growing evidence that supports the hypothesis that cytokines and chemokines released in the local microenvironment promote metastasis and tumor cell proliferation and growth in a specific, coordinated mechanism.

Experiments conducted during the course of development of the present invention demonstrate a role for MCP-1 in prostate cancer migration and proliferation as a mechanism for increased bone metastases. MCP-1 is a novel potent regulator of prostate cancer migration and proliferation at the site of the bone microenvironment.

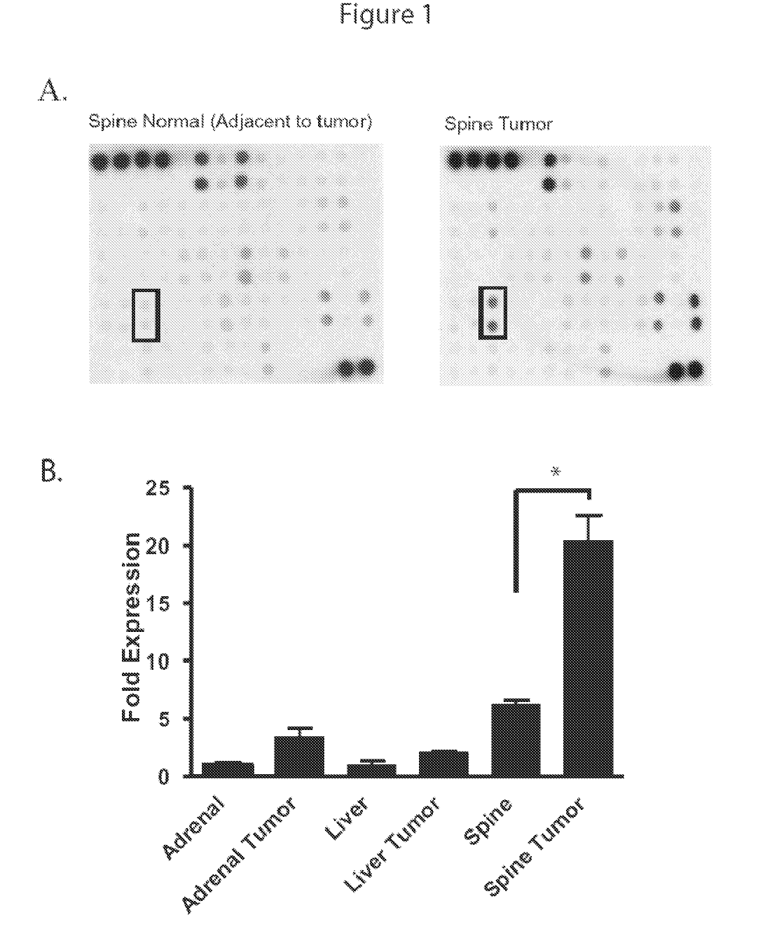

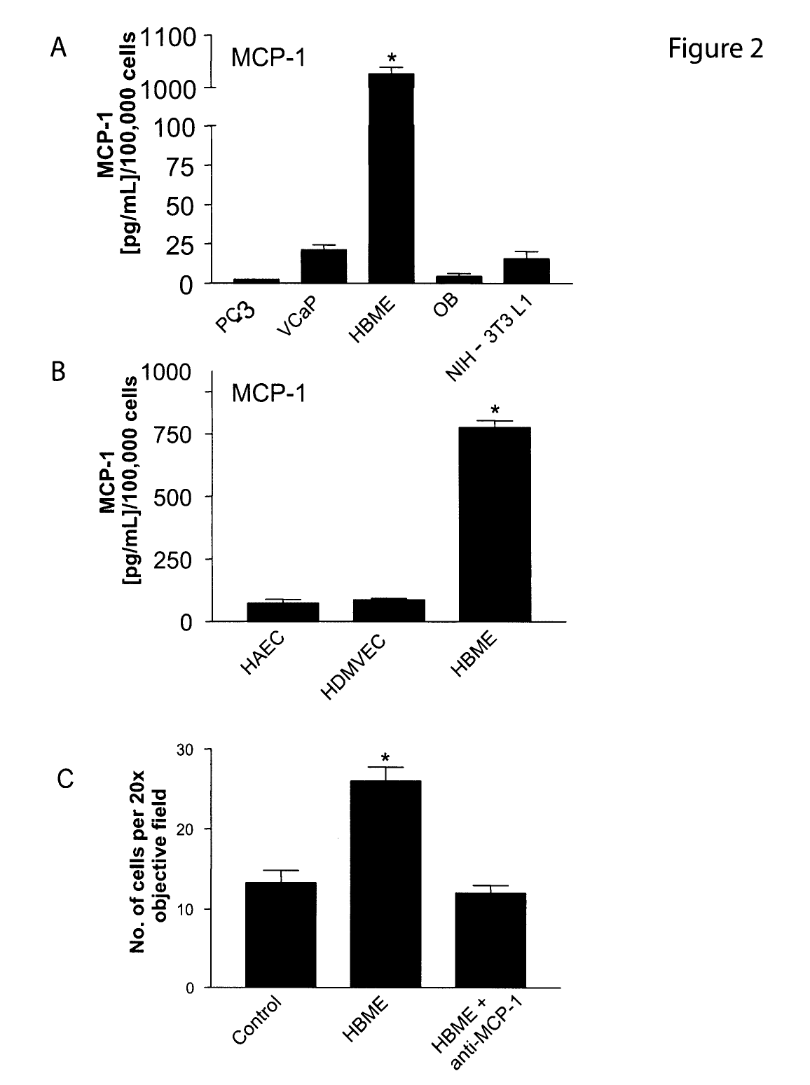

Utilizing tissue procured during the Rapid Autopsy program at the University of Michigan, a comparison was made between cytokine and chemokine expression in the microenvironment of a bone metastasis and normal/adjacent bone using a cytokine/growth factor antibody array (FIG. 1). The most upregulated cytokine in the tumor-bone microenvironment was identified as MCP-1, monocyte chemoattractant protein 1. MCP-1 belongs to a family of cytokines that is known to promote migration of monocytes and macrophages to sites of inflammation. Recently a role of MCP-1 in regulating the migration and proliferation of cancer epithelial cells has been shown in breast cancer and multiple myeloma (Vanderkerken et al., supra; Ohta et al., (2002) Int J Cancer 102, 220-224; Lebrecht et al., (2004) Tumour Biol 25, 14-17; Valkovic et al., (1998) Pathol Res Pract 194, 335-340). Upregulation of cytokines at the site of a secondary lesion has been postulated to play an important role in “homing” and tumor formation. SDF-1/CXCR4 has recently been shown to exert a predominant role in regulating prostate cancer cell metastasis to the bone (Taichman et al., supra). Experiments conducted during the course of development of the present invention demonstrate the ability of MCP-1 to stimulate prostate cancer cell migration and proliferation in a dose-dependent manner. Additionally, the predominant source of MCP-1 in the bone microenvironment is the bone marrow endothelial cells (FIG. 2).

Prior to the present invention, the only study addressing a role of MCP-1 in prostate cancer focused on MCP-1 expression in prostate epithelial cells and stromal cells during benign prostatic hyperplasia and localized prostatic adenocarcinoma. MCP-1 was shown to be expressed by smooth muscle cells in the prostate gland surrounding the epithelial cells and in the benign epithelial cells. MCP-1 expression was reported to be less in the cancerous epithelial cells of localized prostate cancer (Mazzucchelli et al., (1996) Am J Pathol 149, 501-509). The data presented here show that the function of MCP-1 in prostate cancer pathogenesis may be localized to the metastatic process and is associated with an important, novel mechanism of “bone homing” of prostate cancer cells. In experiments conducted during the course of development of the present invention, human bone marrow endothelial cells were shown secrete significantly higher levels of MCP-1 compared to human aortic endothelial cells, as well as human dermal microvascular endothelial cells. This indicates that the bone marrow endothelium plays an important role in regulating prostate cancer metasatsis by secreting MCP-1. Furthermore, a specific role of MCP-1 to act as a chemoattractant for bone derived prostate cancer epithelial cells and regulates their migratory properties in a dose-dependent fashion was demonstrated. Additionally, it was demonstrated that MCP-1 stimulates proliferation of prostate cancer cells in a PI3kinase/Akt dependent mechanism with further downstream activation of the p70 S6 kinase. Activation of p70 S6 kinase has been shown to regulate changes in the actin cytoskeleton (Raymond et al., (2002) Neuroscience 109, 531-536) and, thus, may play a role in the enhanced migratory phenotype of prostate cancer cells when stimulated with MCP-1. The chemokine family has been postulated to play a significant role in tumorigenesis and metastasis of several human cancers (Balkwill, (2003) Semin Immunol 15, 49-55; Balkwill, (2004) Nat Rev Cancer 4, 540-550). Recently, evidence has suggested that CCR2, the high affinity receptor for MCP-1, is linked to the actin cytoskeleton via interactions with FROUNT (Gavrilin et al., (2005) Biochem Biophys Res Commun 327, 533-540; Terashima et al., (2005) Nat Immunol 6, 827-835).

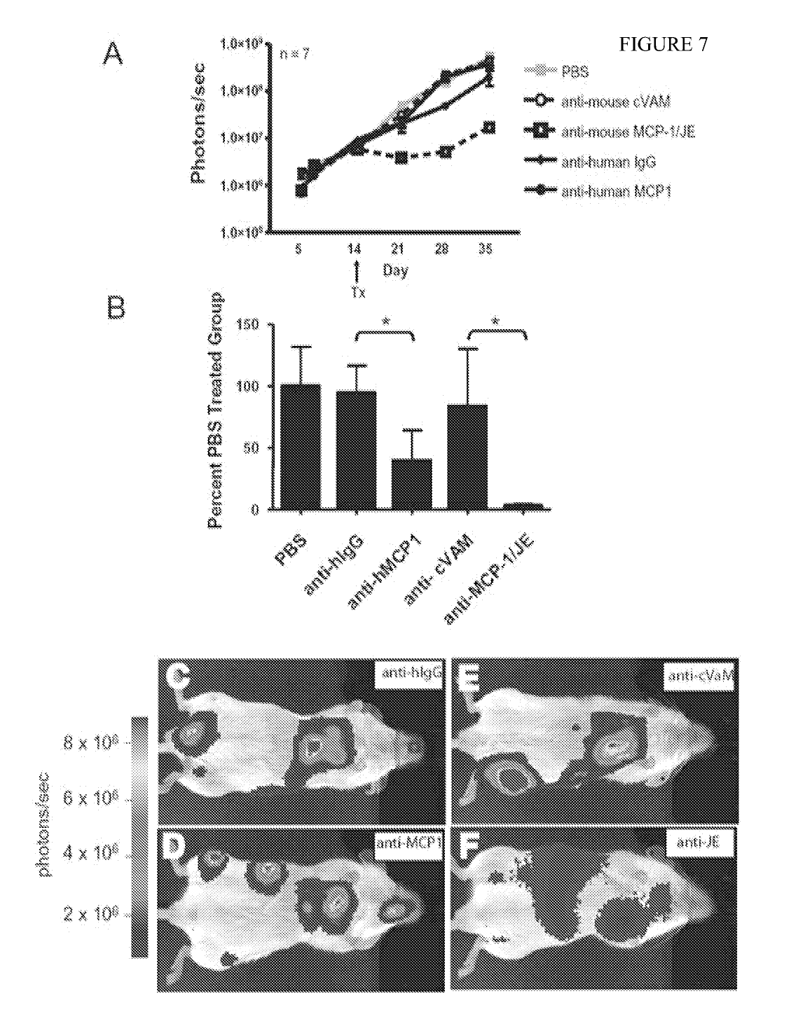

To further investigate the role of MCP-1 in prostate cancer tumorigenesis and metastasis, an in vivo bioluminescent model of metastasis as previously described was utilized (Loberg et al., (2006) Neoplasia 8). PC-3^{Luc} cells were injected by intracardiac injection in mice being treated with neutralizing antibodies to the human MCP-1 and compared them with mice being treated with neutralizing antibodies targeting the mouse MCP-1/JE homolog. Utilizing the anti-human MCP-1 allows one to target MCP-1 being secreted by the tumor cells specifically while the anti-mouse MCP-1/JE targets MCP-1 secreted from the host. Inhibition of MCP-1 with the anti-human MCP-1 antibody resulted in a 46.52% reduction in overall tumor burden compared to the anti-human IgG control antibody group (FIGS. 7b,c&d). Treatment of PC-3^{Luc} injected animals with the anti-mouse MCP-1/JE neutralizing antibody resulted in a 95.91% reduction in overall tumor burden compared to the anti-mouse cVaM control antibody (FIGS. 7b,e&f). These data indicate that MCP-1 secreted from the tumor cell acts in a paracrine/autocrine fashion to promote cell survival and growth while MCP-1 secreted from the host environment, specifically the bone marrow endothelial cells, stimulates migration and proliferation in the bone microenvironment.

Additional experiments conducted during the course of development of the present invention provides data that demonstrates the importance of the chemokine, CCL2 (MCP-1), in prostate cancer bone metastasis using an intratibial model of prostate cancer. CCL2 is released from osteoblasts and bone marrow endothelial cells and is further induced in response to PTHrP stimulation. Several studies have reported that prostate cancer cells secrete high levels of PTHrP and that secretion of PTHrP is important in the establishment of prostate cancer in the bone microenvironment. Tumor-derived PTHrP is known to upregulate receptor activator of NFkappaB ligand (RANKL), an essential factor in osteoclastogenesis. Further, CCL2 has been shown to directly stimulate the proliferation of prostate cancer cells (Loberg et al., Urol. Onc. 2006; 24:161-168). In addition, systemic inhibition of CCL2 with neutralizing antibodies inhibits the growth and establishment of prostate cancer cells in the bone microenvironment. PC-3 cells are known to form purely lytic lesions compared to VCaP cells which are shown here to form a mixture of osteolytic and osteoblastic lesions. Anti-CCL2 neutralizing antibodies partially reduced PC-3 tumor growth and TRAP+ osteoclast activity and completely inhibited osteoclast activation and establishment of VCaP tumors. The present invention is not limited to a particular mechanism. Indeed, an understanding of the mechanism is not necessary to practice the present invention. Nonetheless, it is contemplated that osteolysis is an important early event in the establishment of prostate cancer in the bone compartment. The purely lytic PC-3 cells may retain the ability to establish bone lesions with anti-CCL2 challenge while anti-CCL2 completely inhibited the ability of VCaP cells to establish bone tumors. The decreased tumor growth was accompanied by a decrease in TRAP5b serum levels supporting the inhibition of osteoclast activity by inhibiting CCL2. The present invention is not limited to a particular mechanism. Indeed, an understanding of the mechanism is not necessary to practice the present invention. Nonetheless, it is contemplated that the data presented here supports the hypothesis that CCL2 is important in the regulation of tumor-induced osteoclast activity and is an important target for the treatment of bone-specific disease.

In vivo experiments conducted during the course of development of the present invention demonstrate a role for CCL2 in prostate cancer tumorigenesis and metastasis via at least two distinct pathways: 1) a direct effect on tumor cell growth and function, and 2) an indirect affect on the tumor microenvironment via regulation of macrophage mobilization and infiltration into the tumor bed. The experiments demonstrated that targeting the CCL2 contributed by the human cancer cells attenuates tumor growth, and that CCL2 secreted by the tumor cells contributes to the CCL2-dependent tumor growth via a paracrine/autocrine mechanism since mouse CCL2 is not affected by anti-hCCL2. Additionally, anti-tumor efficacy derived from targeting the mouse CCL2 demonstrates that there is a cooperation between tumor cell-derived CCL2 and host-derived CCL2 promoting tumor cell growth and metastasis (Loberg et al., 2006. Neoplasia. 8:578-586). The present invention is not limited to a particular mechanism. Indeed, an understanding of the mechanism is not necessary to practice the present invention. Nonetheless, it is contemplated that in the current model using SCID mice (T cell and B cell deficient), this effect is through recruitment of host monocytes/macrophages.

The results of experiments conducted during the course of development of the present invention demonstrated that administration of an anti-CCL2 antibody alone or in combination with docetaxel provides a therapeutic strategy for the treatment of prostate cancer. The addition of docetaxel to antibody treatment induced greater tumor regression in vivo compared to docetaxel alone. Continuing animals on antibody therapy after the cessation of docetaxel maintained tumor regression and the development of additional tumor burden compared to animals receiving docetaxel alone.

Accordingly, in some embodiments, the present invention provides methods of treating prostate cancer (e.g., metastatic prostate cancer) and preventing prostate cancer metastasis (e.g., to bone). The present invention further provides methods of diagnosing and characterizing prostate cancer (e.g., identifying cancer that has metastasized or is likely to metastasize). The present invention additionally provides research methods (e.g., drug screening methods) for the identification of new therapeutic agents.

### I. Cancer Therapies

In some embodiments, the present invention provides therapies for cancer (e.g., prostate cancer). In some embodiments, therapies target MCP-1. As described herein, experiments conducted during the course of development of the present invention demonstrated a role for MCP-1 in prostate cancer metastasis to the bone. Further experiments demonstrated that blockage of MCP-1 resulted in a decrease in tumor proliferation in vivo. Accordingly, in some embodiments, the present invention provides methods of treating prostate cancer (e.g., metastatic prostate cancer). In other embodiments, the present invention provides methods of preventing prostate cancer metastasis.

A. Antibody Therapy

In some embodiments, the present invention provides antibodies that target prostate tumors that express MCP-1. Any suitable antibody (e.g., monoclonal, polyclonal, or synthetic) may be utilized in the therapeutic methods disclosed herein. In some embodiments, antibodies are identified using the drug screening methods of the present invention. In other embodiments, antibodies described in Examples 1 and 2 below are utilized. In some embodiments, antibodies are antibodies to human MCP-1 (e.g., CNTO888). In other embodiments, antibodies are antibodies to a mouse (or other animal) MCP-1 homolog (e.g., C1142). In yet other embodiments, antibodies known in the art (See e.g., WO 04/080273, WO 04/050836, WO 04/016769 and U.S. application 2006 0039913, each of which is herein incorporated by reference in its entirety) are utilized.

In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against MCP-1, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted MCP-1. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

B. Antisense Therapies

In some embodiments, the present invention targets the expression MCP-1. For example, in some embodiments, the present invention employs compositions comprising oligomeric antisense compounds, particularly oligonucleotides (e.g., those identified in the drug screening methods described above), for use in modulating the function of nucleic acid molecules encoding MCP-1, ultimately modulating the amount of MCP-1 expressed. This is accomplished by providing antisense compounds that specifically hybridize with one or more nucleic acids encoding MCP-1. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of MCP-1. In the context of the present invention, “modulation” means either an increase (stimulation) or a decrease (inhibition) in the expression of a gene. For example, expression may be inhibited to potentially prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding MCP-1. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as—O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]2, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. degree ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-5-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

C. RNA Interference (RNAi)

In other embodiments, RNAi is utilized to inhibit MCP-1 function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Corners, synthesised using a physical barrier (mask) by stepwise addition of each base in the sequence. Thus the arrays represent a full oligonucleotide complement of a region of the target gene. Hybridisation of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol. Biol. 2005 May 13; 348(4):883-93, J Mol. Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

D. Genetic Therapies

The present invention contemplates the use of any genetic manipulation for use in modulating the expression of MCP-1. Examples of genetic manipulation include, but are not limited to, gene knockout (e.g., removing the MCP-1 gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

E. Small Molecules

In still further embodiments, the present invention provides drugs (e.g., small molecule drugs) that target MCP-1 activity. In some embodiments, small molecule drugs are identified using the drug screening methods described below. In other embodiments, small molecule drugs are described in WO 04/071460, WO 04/071499, WO 03/084993, WO 03/075853, WO 05/021500, WO 05/021499, U.S. Applications 20040171552 and 20040138171, WO 03/072599, WO 05/021498, WO 05/020899, WO 04/098516 and WO 04/098512, each of which is herein incorporated by reference in its entirety.

F. TRAP Compositions

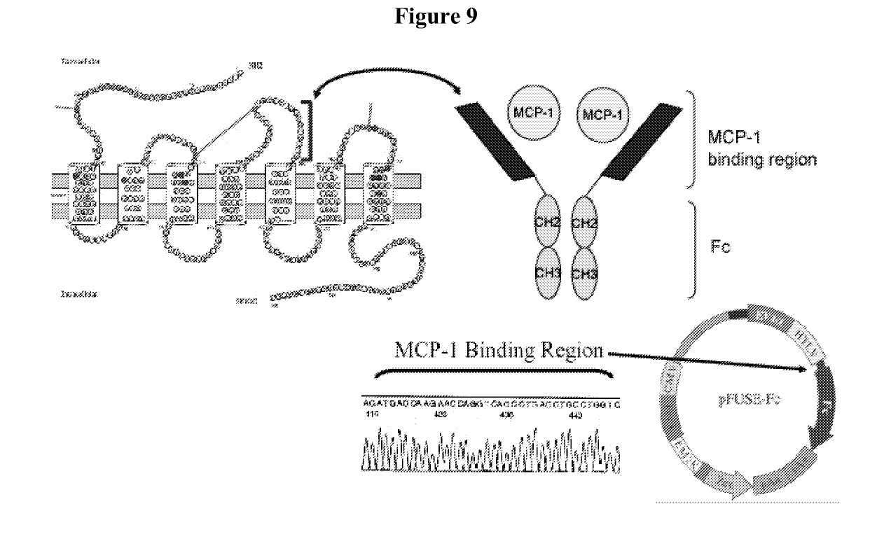

In some further embodiments, the present invention provides MCP-1 TRAP molecule. In some embodiments, the TRAP is an Fc fusion protein similar to the VEGF TRAP molecules recently published (Holash et al., Proc Natl Acad Sci USA, 2002. 99(17): p. 11393-8). Utilizing the known binding sequence of the high affinity receptor for MCP-1 (WNNFHTIMR), experiments conducted during the course of development of the present invention generated an MCP-1 TRAP molecule. In vitro use of an antagonist binding peptide yielded a binding affinity of 2.20±0.44×10−5 M (Kim et al., FEBS Lett, 2005. 579(7): p. 1597-601). Synthesis of an MCP-1R TRAP molecule by coupling the binding sequence of the high affinity receptor to the Fc fragment of the human IgG immunoglobulin, as described for the VEGF TRAP molecule, will extend plasma half life (t ½) for use during in vivo models of metastasis, as well as providing an efficient, extremely high affinity, fast method of designing targeting MCP-1 (FIG. 9 and Example 3). For example, pharmacokinetics of the VEGF TRAPR1R2 molecule demonstrated a superior profile to comparable neutralizing VEGF antibodies with a Cmax of 16 μg/mL, an AUC of 36.28 μg×days/mL and retained a 1 μM binding affinity for VEGF165 (Holash et al., supra). Thus, it is contemplated that MCP-1 TRAP molecules are an extremely sensitive, useful tool in dissecting the importance of MCP-1 in prostate cancer metastasis and proliferation, as well as in therapeutic applications.

G. Combination Therapy

In still further embodiments, one or more of the above described therapeutic agents are administered in combination. Experiments conducted during the course of development of the present invention (Example 4 below) demonstrated that a combination of TAXOTERE and an antibody directed towards MCP-1 was more effective in reducing tumor burden in mice than either agent alone. Accordingly, in some embodiments, a combination of a known chemotherapy agent (e.g., TAXOTERE) and an antibody directed towards MCP-1 are utilized in the treatment of prostate cancer. In certain embodiments, combination therapy (e.g., using an MCP-1 antibody and a known chemotherapy agent) is initially utilized, followed by maintenance therapy with a single agent (e.g., an antibody directed toward MCP-1).

In some embodiments, the compounds of the present invention are provided in combination with known cancer chemotherapy agents. The present invention is not limited to a particular chemotherapy agent.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in compositions and methods of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 3 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 3

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and |
| (IgG1κ anti CD52 antibody) | | ILEX Partners, LP, |
| | | Cambridge, MA |
| Alitretinoin | Panretin | Ligand |
| (9-cis-retinoic acid) | | Pharmaceuticals, Inc., |
| | | San Diego CA |
| Allopurinol | Zyloprim | GlaxoSmithKline, |
| (1,5-dihydro-4H-pyrazolo[3,4-d]pyrimidin-4-one | | Research Triangle |
| monosodium salt) | | Park, NC |
| Altretamine | Hexalen | US Bioscience, West |
| (N,N,N′,N′,N″,N″,-hexamethyl-1,3,5-triazine-2,4, | | Conshohocken, PA |
| 6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile,a,a,a′,a′-tetramethyl- | | Pharmaceuticals, LP, |
| 5-(1H-1,2,4-triazol-1-ylmethyl)) | | Wilmington, DE |
| Arsenic trioxide | Trisenox | Cell Therapeutic, |
| | | Inc., Seattle, WA |
| Asparaginase | Elspar | Merck & Co., Inc., |
| (L-asparagine amidohydrolase, type EC-2) | | Whitehouse Station, |
| | | NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| [BCG], substrain Montreal) | | |
| bexarotene capsules | Targretin | Ligand |
| (4-[1-(5,6,7,8-tetrahydro-3,5,5,8,8-pentamethyl-2- | | Pharmaceuticals |
| napthalenyl) ethenyl] benzoic acid) | | |
| bexarotene gel | Targretin | Ligand |
| | | Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle |
| (as 4-[5-(4-methylphenyl)-3-(trifluoromethyl)- | | Pharmaceuticals, |
| 1H-pyrazol-1-yl] | | England |
| benzenesulfonamide) | | |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R. W. Johnson |
| (2-chloro-2′-deoxy-b-D-adenosine) | | Pharmaceutical |
| | | Research Institute, |
| | | Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye |
| | | Pharmaceuticals, Inc., |
| | | San Diego, CA |
| Dacarbazine | DTIC-Dome | Bayer AG, |
| (5-(3,3-dimethyl-1-triazeno)-imidazole-4- | | Leverkusen, |
| carboxamide (DTIC)) | | Germany |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., |
| (recombinant peptide) | | Thousand Oaks, CA |
| daunorubicin liposomal | DanuoXome | Nexstar |
| ((8S-cis)-8-acetyl-10-[(3-amino-2,3,6-trideoxy-a- | | Pharmaceuticals, Inc., |
| L-lyxo-hexopyranosyl)oxy]-7,8,9,10-tetrahydro- | | Boulder, CO |
| 6,8,11-trihydroxy-1-methoxy-5,12- | | |
| naphthacenedione hydrochloride) | | |
| Daunorubicin HCl, daunomycin | Cerubidine | Wyeth Ayerst, |
| ((1S,3S)-3-Acetyl-1,2,3,4,6,11-hexahydro- | | Madison, NJ |
| 3,5,12-trihydroxy-10-methoxy-6,11-dioxo-1- | | |
| naphthacenyl 3-amino-2,3,6-trideoxy-(alpha)-L- | | |
| lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one 4- | | |
| acetate 2-benzoate, trihydrate) | | |
| Doxorubicin HCl | Adriamycin, | Pharmacia & Upjohn |
| (8S,10S)-10-[(3-amino-2,3,6-trideoxy-a-L-lyxo- | Rubex | Company |
| hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin PFS | Pharmacia & Upjohn |
| | Intravenous | Company |
| | injection | |
| doxorubicin liposomal | Doxil | Sequus |
| | | Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10-tetrahydro- | | |
| 6,8,11-trihydroxy-8-(hydroxyacetyl)-1-methoxy- | | |
| 5,12-naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-,3- | | Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside],4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, |
| agent vidarabine, 9-b-D-arabinofuranosyladenine | | NJ |
| (ara-A)) | | |
| Fluorouracil, 5-FU | Adrucil | ICN Pharmaceuticals, |
| (5-fluoro-2,4(1H,3H)-pyrimidinedione) | | Inc., Humacao, |
| | | Puerto Rico |
| Fulvestrant | Faslodex | IPR Pharmaceuticals, |
| (7-alpha-[9-(4,4,5,5,5-penta fluoropentylsulphinyl) | | Guayama, Puerto |
| nonyl]estra-1,3,5-(10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of [D-Ser(But)⁶,Azgly¹⁰]LHRH; pyro- | | Pharmaceuticals |
| Glu-His-Trp-Ser-Tyr-D-Ser(But)-Leu-Arg-Pro- | | |
| Azgly-NH2 acetate [C₅₉H₈₄N₁₈O₁₄•(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator tiuxetan [N- | | |
| [2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione,9-acetyl-7-[(3-amino- | | Company |
| 2,3,6-trideoxy-(alpha)-L-lyxo- | | |
| hexopyranosyl)oxy]-7,8,9,10-tetrahydro-6,9,11- | | |
| trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4-methyl- | | Switzerland |
| 3-[[4-(3-pyridinyl)-2-pyrimidinyl]amino]- | | |
| phenyl]benzamide methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′,4′: | | |
| 6,7] indolizino[1,2-b] quinoline-3,14(4H, 12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-(S)-2,3,5,6-tetrahydro-6-phenylimidazo [2,1- | | Foundation, |
| b] thiazole monohydrochloride C₁₁H₁₂N₂S•HCl) | | Titusville, NJ |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7-one) | | Exton, Pa |
| Mitomycin C | Mutamycin | Bristol-Myers Squibb |
| mitomycin C | Mitozytrex | SuperGen, Inc., |
| | | Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, |
| | | Germany |
| Oprelvekin | Neumega | Genetics Institute, |
| (IL-11) | | Inc., Alexandria, VA |
| Oxaliplatin | Eloxatin | Sanofi Synthelabo, |
| (cis-[(1R,2R)-1,2-cyclohexanediamine-N,N′] | | Inc., NY, NY |
| [oxalato(2-)-O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate 2- | | |
| benzoate 13-ester with (2R, 3 S)-N-benzoyl-3- | | |
| phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| ((monomethoxypolyethylene glycol succinimidyl) | (Pegademase | Pharmaceuticals, Inc., |
| 11-17-adenosine deaminase) | Bovine) | Bridgewater, NJ |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p-toluamide | | Pharmaceuticals, Inc., |
| monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, |
| (recombinant peptide) | | Inc., |
| Rituximab | Rituxan | Genentech, Inc., |
| (recombinant anti-CD20 antibody) | | South San Francisco, |
| | | CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and b)- | | |
| D-glucopyranose and 220 mg citric acid | | |
| anhydrous) | | |
| Talc | Sclerosol | Bryan, Corp., |
| (Mg₃Si₄O₁₀(OH)₂) | | Woburn, MA |
| Tamoxifen | Nolvadex | AstraZeneca |
| ((Z)2-[4-(1,2-diphenyl-1-butenyl) phenoxy]-N,N- | | Pharmaceuticals |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| Teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien- | | |
| 17-oic acid [dgr]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex |
| (Aziridine, 1,1′,1″-phosphinothioylidynetris-, or | | Corporation |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′:6,7] indolizino [1,2-b] | | |
| quinoline-3,14-(4H,12H)-dione | | |
| monohydrochloride) | | |
| Toremifene | Fareston | Roberts |
| (2-(p-[(Z)-4-chloro-1,2-diphenyl-1-butenyl]- | | Pharmaceutical |
| phenoxy)-N,N-dimethylethylamine citrate (1:1)) | | Corp., Eatontown, NJ |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2-oxoethyl | | |
| pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

H. Pharmaceutical Compositions

The present invention further provides pharmaceutical compositions (e.g., comprising the therapeutic compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### II. Markers for Prostate Cancer

The present invention further provides markers whose expression is specifically altered in cancerous prostate tissues. Such markers find use in the diagnosis and characterization of prostate cancer. For example, in some embodiments, increased levels of MCP-1 in prostate samples serve as an indicator of the presence of cancer or the presence of cancer that has metastasized or is likely to metastasize (e.g., to the bone).

In some embodiments, the present invention provides methods for detection of expression of MCP-1. In preferred embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, prostatic secretions, and urine). The present invention further provides panels and kits for the detection of markers. In preferred embodiments, the presence of a cancer marker (e.g., MCP-1) is used to provide a prognosis to a subject. For example, the detection of increased levels of expression of MCP-1 in prostate samples is associated with tumors that have metastasized. The information provided is also used to direct the course of treatment. For example, if a subject is found to have a marker indicative of a highly metastasizing tumor, additional therapies (e.g., hormonal or radiation therapies) can be started at a earlier point when they are more likely to be effective (e.g., before metastasis).

1. Detection of RNA

In some preferred embodiments, detection of MCP-1 is detected by measuring the expression of corresponding mRNA in a tissue sample (e.g., prostate tissue). mRNA expression may be measured by any suitable method, including but not limited to, those disclosed below.

In some embodiments, RNA is detection by Northern blot analysis. Northern blot analysis involves the separation of RNA and hybridization of a complementary labeled probe. An exemplary method for Northern blot analysis is provided in Example 3.

In still further embodiments, RNA (or corresponding cDNA) is detected by hybridization to a oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

2. Detection of Protein

In other embodiments, gene expression MCP-1 is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to MCP-1 is utilized.

In other embodiments, the immunoassay described in U.S. Pat. Nos. 5,599,677 and 5,672,480; each of which is herein incorporated by reference.

3. Data Analysis

In some embodiments, a computer-based analysis program is used to translate the raw data generated by the detection assay (e.g., the presence, absence, or amount of MCP-1) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of metastasis) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

4. Kits

In yet other embodiments, the present invention provides kits for the detection and characterization of prostate cancer. In some embodiments, the kits contain antibodies specific for a MCP-1, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

5. In Vivo Imaging

In some embodiments, in vivo imaging techniques are used to visualize the expression of MCP-1 in an animal (e.g., a human or non-human mammal). For example, in some embodiments, MCP-1 is labeled using an labeled antibody specific for MCP-1. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to MCP-1 are described below.

The in vivo imaging methods of the present invention are useful in the diagnosis of cancers that express MCP-1 (e.g., prostate cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for MCP-1 are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nuc. Med. Biol 17:247-254 [1990] have described an optimized antibody-chelator for the radioimmunoscintographic imaging of tumors using Indium-111 as the label. Griffin et al., (J Clin One 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of MCP-1, to insure that the antigen binding site on the antibody will be protected. The antigen is separated after labeling.

In still further embodiments, in vivo biophotonic imaging is utilized for in vivo imaging. This real-time in vivo imaging utilizes luciferase. The luciferase gene is incorporated into cells, microorganisms, and animals (e.g., as a fusion protein with MCP-1). When active, it leads to a reaction that emits light. A CCD camera and software is used to capture the image and analyze it.

### III. Antibodies

The present invention provides isolated antibodies. In preferred embodiments, the present invention provides monoclonal antibodies that specifically bind to an isolated polypeptide comprised of at least five amino acid residues of MCP-1. These antibodies find use in the diagnostic and therapeutic methods described herein. Exemplary antibodies are described in the above sections and in Examples 1 and 2 or can be generated using the methods described below.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against MCP-1) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, MCP-1 (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### IV. Drug Screening

In some embodiments, the present invention provides drug screening assays (e.g., to screen for anticancer drugs). In some embodiments, the screening methods of the present invention utilize MCP-1. For example, in some embodiments, the present invention provides methods of screening for compound that alter (e.g., increase or decrease) the expression of MCP-1. In some embodiments, candidate compounds are antisense or siRNA agents (e.g., oligonucleotides) directed against MCP-1. In other embodiments, candidate compounds are antibodies that specifically bind to MCP-1. In yet other embodiments, candidate compounds are small molecules that inhibit a biological activity of MCP-1.

In one screening method, candidate compounds are evaluated for their ability to alter MCP-1 expression by contacting a compound with a cell expressing MCP-1 and then assaying for the effect of the candidate compounds on expression. In some embodiments, the effect of candidate compounds on expression of MCP-1 is assayed for by detecting the level of MCP-1 mRNA expressed by the cell. mRNA expression can be detected by any suitable method.

In other embodiments, the effect of candidate compounds on expression of MCP-1 is assayed by measuring the level of MCP-1 polypeptide. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to MCP-1, have an inhibitory effect on, for example, MCP-1 expression or MCP-1 activity, or have a stimulatory or inhibitory effect on, for example, the expression or activity of a MCP-1 substrate. Compounds thus identified can be used to modulate the activity of target gene products (e.g., MCP-1) either directly or indirectly in a therapeutic protocol, to elaborate the biological function of the target gene product, or to identify compounds that disrupt normal target gene interactions. Compounds which inhibit the activity or expression of MCP-1 are useful in the treatment of proliferative disorders, e.g., cancer, particularly metastatic (e.g., to the bone) prostate cancer.

In one embodiment, the invention provides assays for screening candidate or test compounds that are substrates of MCP-1 protein or polypeptide or a biologically active portion thereof. In another embodiment, the invention provides assays for screening candidate or test compounds that bind to or modulate the activity of MCP-1 protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J., Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

This invention further pertains to novel agents identified by the above-described screening assays. Accordingly, it is within the scope of this invention to further use an agent identified as described herein (e.g., a MCP-1 modulating agent, an antisense MCP-1 nucleic acid molecule, a siRNA molecule, a MCP-1 specific antibody, or a MCP-1-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### V. Transgenic Animals Expressing MCP-1 Genes

The present invention contemplates the generation of transgenic animals that over-express or under-express (e.g., knockout animals) MCP-1.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

### EXPERIMENTAL

The following examples are provided in order to demonstrate and further illustrate certain preferred embodiments and aspects of the present invention and are not to be construed as limiting the scope thereof.

### Example 1

### Role of MCP-1 in Prostate Cancer Metastasis

This Example describes the role of MCP-1 in prostate cancer metastasis to the bone.

### A. Experimental Procedures

Materials-Human recombinant MCP-1 and anti-MCP-1 antibody were obtained from Chemicon International (Temecula, Calif.), anti-phospho Akt_{Ser473} and anti-Akt were obtained from Cell Signaling (Beverly, Mass.), all other reagents were obtained from Sigma-Aldrich.

Cell Culture-PC-3, VCaP, HAEC, HMVEC, HBME were obtained from ATCC and passaged under appropriate growth conditions. PC-3 cells were maintained in RPMI 1640+10% Fetal Calf Serum (FCS) (Invitrogen Corp.). HAEC and HMVEC cells were maintained in EGM+5% FCS while VCaP and HBME cells were maintained in DMEM (Invitrogen Corp.). Cells were passaged by trypsinization using 1× Trypsin+EDTA (Invitrogen Corp.) and resuspended in appropriate growth media.

Cytokine Antibody Array-Normal vertebral and tumor vertebral tissue was collected from a patient with advanced hormone refractory prostate cancer in accordance with the Rapid Autopsy series conducted at the University of Michigan. Tissue specimens were snap frozen in liquid nitrogen and pulverized with a mortor and pestal. Crushed tissue was resuspended in RIPA buffer (50 mM Tris-HCl, pH 7.4, 1% NP-40, 150 mM NaCl, 1 mM EDTA) containing protease inhibitors [1 μg/mL aprotinin, leupeptin and pepstatin A, 1 mM PMSF, 1 mM NaF, and 1 mM Na₃VO₄]. Lysates were pulsed sonicated at 40% duty cycle for 5 sec and protein lysates were collected by centrifugation at 13,000 rpm for 15 min at 4 C. Protein lysates were diluted and cytokine arrays were performed according to the manufacturer's instructions (RayBiotech, Inc.).

MCP-1 ELISA-Cells were plated in 6-well plates and grown to 80% confluency in appropriate growth media. Cells were then washed with serum-free RPMI 1640 or DMEM supplemented with 1% penicillin and streptomycin. Cells were incubated in serum free media for 24 hours and conditioned medium from each well was collected and stored at −80° C. until use. The level of MCP-1 in cell culture supernatants was determined by using QUANTIKINE human MCP-1 sandwich ELISA kit from R&D Systems (Minneapolis, Minn.) according to the protocol supplied by the manufacturers.

Boyden Chamber Migration Assay—HBME conditioned media was collected as described above and used as the chemoattractant in the lower chamber of a modified Boyden Chamber. Cells were harvested by 0.5 μM EDTA release and resuspended in serum free media at 5×10⁴ cells/ml. 2.5×10⁴ cells were added to the upper chamber of the transwell insert and incubated for 24 hours at 37° C. and 5% CO₂ atmosphere. At the end of the incubation period, the cells were fixed with 4% formaldehyde in PBS for 5 minutes. Non-adherent cells were removed from inside the inserts with cotton tipped swab. Cells which had migrated to the underside of the insert were stained with 0.5% crystal violet for 5 minutes and rinsed thoroughly with tap water. Inserts were allowed to dry and the cells were counted using an inverted microscope.

Flouresence-based Migration Assay—Cell migration was assessed using the Innocyte Cell Migration Assay (Calbiochem, Inc.) following the manufacturer's instructions. Briefly, increasing concentrations of MCP-1 (1-100 ng/mL) in the presence and absence of CCR2 inhibitors or neutralizing antibodies were added to the lower chamber of a 96 well plate. Cells were harvested by 0.5 μM EDTA release and resuspended at 2.5×10⁵ cells/mL in serum free media. 2.5×10⁴ cells were added to the upper chamber and allowed to migrate through the membrane with 8 μm pores for 24 hours at 37° C. and 5% CO₂ atmosphere. Cells that migrated through the membrane were detached and labeled with Calcein AM and fluorescence was measured using fluorescent plate reader with excitation wavelength of 485 nm and emission wavelength of 520 nm. The experiments were repeated twice and each conditioned was performed in quadruplicate in each experiment.

Western Blot-Cells were lysed in RIPA buffer (50 mM Tris-HCl, pH 7.4, 1% NP-40, 150 mM NaCl, 1 mM EDTA, 1 mM PMSF, 1 mM Na₃VO₄, 1 mM NaF, 1 uM okadaic acid and 1 ug/mL aprotinin, leupeptin and pepstatin). Proteins were separated under reducing conditions by SDS-PAGE and transferred onto nitrocellulose membrane. The membranes were blocked with 5% milk in TBST (0.1% Tween in TBS) for 1 hour at room temperature. They were incubated overnight at 4° C. with primary antibodies. Membranes were washed 3 times prior to incubation with HRP-conjugated secondary antibodies (Cell Signaling, Beverly, Mass.) for 1 hour at room temperature. Protein expression was visualized by ECL chemiluminescent (Promega, Madison, Wis.) and quantitated using Image J software (NCl, Bethesda, Md.).

Immunofluorescence-PC-3 cells were plated on glass coverslips and serum starved for 2 hours prior to stimulation. Cells were stimulated with MCP-1 (100 ng/mL) for 30 min in the presence or absence of identified inhibitors. Stimulation with SDF-1 (200 ng/mL) for 30 min was used as a positive control. Cells were fixed in 3.7% paraformaldehyde, methanol-free for 10 min at room temperature then permeablized with 0.5% Triton X-100 for 5 min at room temperature. Cells were rinsed twice with PBS and incubated for 30 minutes at room temperature with 3% bovine serum albumin (BSA) in PBS+0.05% Tween 20 to prepare cells for staining. Cells were incubated with Phalloidin AlexaFluor S68 (Molecular Probes, Inc.) at a 1:40 dilution in BSA solution to label actin. Cells were washed and mounted on coverslips with Pro-Long Antifade containing DAPI (Molecular Probes, Inc.) following manufacturer's instructions. Immunofluoresence was visualized using a multi-photon laser scanning microscope (MPLSM), consisting of a Mai Tai Broadband Ti:Sapphire laser, tuneable between 710-990 nm and a modified Olympus Fluoview 300 confocal microscope with long working distance water immersion IR objectives. Images were captured with a 60× objective (zoom ×2), 800 nm excitation wavelength, 605 nm and 520 nm centered emmission filters.

Proliferation Assay-Cells were seeded at a density of 4×10³ cells/well in a six well plate in regular media. Twenty four hours after seeding the media was changed with serum free media with increasing concentrations of MCP-1(1-100 ng/mL) in the presence of absence of LY294002 (1 μM). Cell growth was determined at 24, 48, 72, and 96 hours after seeding using the WST-1 assay (Pierce Biotech) following the manufacturer's instructions.

Real time RT-PCR-Total RNA was isolated from cell lines using Trizol (Invitrogen Corp, Carlsbad, Calif.) following the manufacturer's specifications. Purified RNA (5 μg) was converted to cDNA using Super Script II reverse transcriptase (Invitrogen Corp.) following the manufacturer's instructions and used for gene expression analysis by real time PCR using an ABI Prism 7900HT thermocycler. Primers and probes were purchased from Applied Biosystems, Inc. and used with TAQMAN Universal PCR Master Mix, No AMPERASE UNG. GAPDH was used as an internal control to normalize and compare each sample. Cycle conditions for real time PCR were 95° C. (15 sec), 60° C. (1 min), 72° C. (1 min) for 40 cycles. Threshold cycle number for each sample was normalized to GAPDH for that sample and expressed on a log scale relative to GAPDH expression.

Bioluminescent in vivo model of metastatsis—Bioluminescent imaging of PC-3^{Luc} was preformed as previously described through The University of Michigan Small Animal Imaging Resource facility (MSAIR) (Kalikin 2003). Briefly, PC-3Luc cells were introduced into male SCID mice (5-6 wks) by intracardiac injections. Mice were serially imaged weekly for 6 weeks using a CCD IVIS™ system using a 50 mm lens (Xenogen Corp, Alameda, Calif.) and the results were analyzed using LIVINGIMAGE software (Xenogen Corp.). Mice were separated into one of five groups; 1) PBS control, 2) anti-hIgG control human antibody, 3) anti-cVaM control mouse antibody, 4) anti-MCP1 (human), 5) anti-MCP1/JE (mouse). Treatment began at week 2 post-intracardiac injection and mice received 2 mg/Kg antibody twice weekly by intraperitoneal injection. Mice were injected with luciferin (40 mg/mL) intraperitoneally and ventral images were acquired 15 min post-injection under 1.75% isofluorane/air anesthesia. Total tumor burden of each animal was calculated using regions of interest (ROIs) that encompassed the entire animal. Animals were sacrificed after week 6 image and individual organs were harvested and immediately placed in formalin.

Statistics-Data was analyzed with GraphPad Prizm software. A One-way-ANOVA analysis was used with Bonferroni's post-hoc analysis for comparison between multiple groups. A Students T-test was used for comparison between two groups. Significance was defined as a p value <0.05.

### B. Results

### Identification of MCP-1 Expression in the Bone-Tumor Microenvironment

Identification of the prominent cytokines and growth factors involved in the tumor-bone microenvironment is essential to the understanding of prostate cancer metastasis. Specimens were collected from a patient diagnosed with prostatic adenocarcinoma (Gleason 4+4=8, T2c tumor) who initially received radiation therapy. After biochemical failure, this patient was placed on androgen deprivation therapy (Lupron) followed by multiple regimens of chemotherapy for hormone refractory disease. Sites of metastasis were identified by gross examination and tissue samples were collected and snap frozen for analysis. Tumor and normal (adjacent to tumor) bone specimens were collected from L3 vertebrate and processed for total protein lysates. Analysis of cytokine and growth factor expression was performed using cytokine antibody arrays from RayBiotech, Inc. Several cytokines were shown to be upregulated in the tumor-bone microenvironment compared to the normal (adjacent to tumor)-bone microenvironment (FIG. 1a). In particular, MCP-1 (monocyte chemoattractant protein 1) was upregulated 4 fold in the tumor microenvironment compared to normal (FIG. 1b).

### Identification of the Source of MCP-1 from Constituents of the Bone Microenvironment

MCP-1 is known to be a potent stimulator of monocyte and macrophage migration to sites of inflammation (Ohta et al., (2002) Int J Cancer 102, 220-224). To identify a role of MCP-1 in prostate cancer metastasis, MCP-1 secretion was determined by ELISA from PC-3, VCaP, HBME, osteoblasts, and NIH 3T3 L1 adipocytes. Prostate cancer cells were not a significant source of MCP-1, however, the human bone marrow endothelial cells secreted significantly more MCP-1 compared to the other cell lines analyzed (PC-3 2.435±0.123; VCaP 21.037±3.213; HBME 1269.083±26.281; OB 4.32±1.85; adipocytes 18.398±3.874 pg/mL) (mean ±SD) (FIG. 2a). Next, the secretion of MCP-1 from different endothelial cell lines was compared to assess the specificity of HBMEs as a significant source of MCP-1. HAEC (human aortic endothelial cells) and HDMVEC (human dermal microvascular endothelial cells) were used for comparison. HBME cells secreted significantly higher levels of MCP-1 compared to HAEC and HDMVEC cells (HBME 805.26±29.81; HAEC 10.12±3.70; HDMVEC 21.86±8.61 pg/mL) (mean ±SD) (FIG. 2b). In order to identify MCP-1 as an important chemotactic factor secreted by HBME cells that induces prostate cancer cell migration, cells were placed in a modified Boyden Chamber. The number of cells that migrated over a 24 hour period was reported as cells per 20× objective field. Conditioned media from HBME cells was used as the chemoattractant and PC-3 cell migration was measured after a 24 hour period. Conditioned media from HBME cells stimulated the migration of PC-3 cells and the migration was inhibited by the presence of an anti-MCP-1 neutralizing antibody (PC-3: control 11.67±3.06; HBME CM 26.0±1.73; anti-MCP-1 13.33±1.528; mean ±SD) (FIG. 2c).

### Effects of MCP-1 on Prostate Cancer Cell Migration

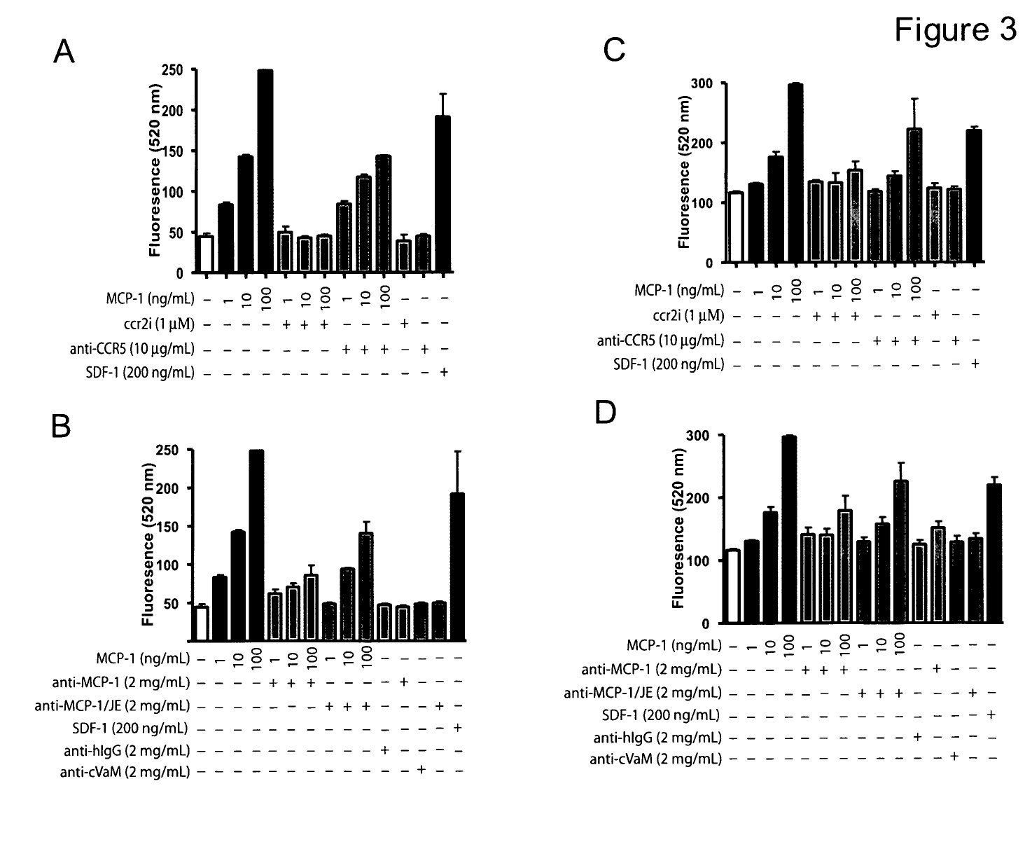

Migration is an essential step in the metastatic cascade and is dependent on the reorganization of the actin cytoskeleton. The majority of data suggests that migration is regulated in part by chemotactic gradients, which stimulate the recruitment of tumor cells to sites of metastases. In prostate cancer, SDF-1 has been postulated as an important chemotactic factor that stimulates prostate cancer cell migration via activation of the CXCR4 receptor (Taichman et al., (2002) Cancer Res 62, 1832-1837). To further the understanding of the role of MCP-1 in prostate cancer cell migration, a 96 well migration assay was used with increasing concentrations of human recombinant MCP-1 (hrMCP-1) as the chemoattractant. PC-3 and VCaP cells migrated in a dose-dependent manner towards hrMCP-1 (PC-3: control 44.62±3.83, 1 ng/mL 83.53±2.981, 10 ng/mL 142.2±2.678, 100 ng/mL 248.1±0.761; VCaP: control 116.4±2.529, 1 ng/mL 130.6±2.145, 10 ng/mL 176.1±9.051, 100 ng/mL 296.5±2.681; mean fluoresence±SD) (FIGS. 3a,c respectively). The dose dependent migration of both PC-3 (FIGS. 3a&b) and VCaP (FIGS. 3a&b) was attenuated by Rs-102895, a CCR2b receptor antagonist (FIGS. 3a&c). MCP-1 induced migration was partially attenuated with the presence of an anti-CCR5 neutralizing antibody (FIGS. 3a&c). Further, the migration of both PC-3 and VCaP cells to MCP-1 at all concentrations was attenuated by the administration of anti-human MCP-1 and anti-mouse MCP-1/JE neutralizing antibodies (FIGS. 3b&d).

### MCP-1 Induces Akt Activation in PC-3 and VCaP Cells

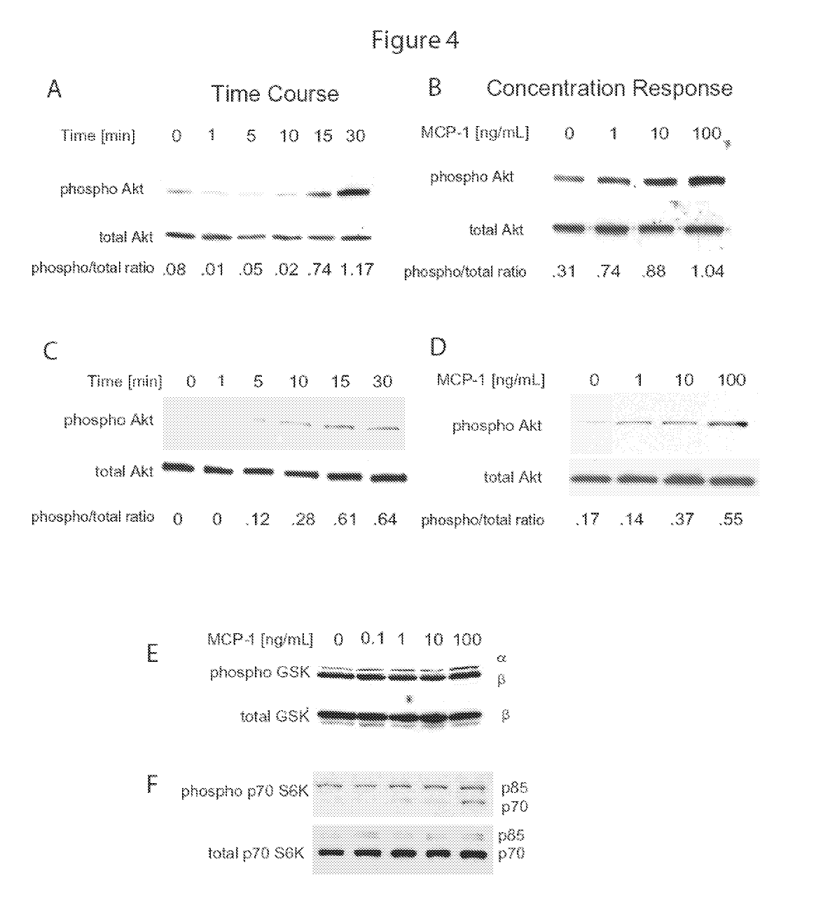

MCP-1 has been shown to induce activation of the PI3kinase/Akt signaling pathway (Choi et al., (2004) FEBS Lett 559, 141-144). To determine the if MCP-1 stimulation of PC-3 and VCaP cells induces similar signaling pathways, PC-3 and VCaP cells were stimulated with a supraphysiological dose of MCP-1 (100 ng/mL) for various time points indicated. MCP-1 induced Akt phosphorylation as measured by immunoblot analysis in a time dependent fashion in both VCaP and PC-3 cells with a maximal activation at 30 min (FIGS. 4a&c). Further, PC-3 and VCaP cells were stimulated with increasing concentrations of MCP-1 (0.1-100 ng/mL) for 30 min. MCP-1 stimulated Akt phosphorylation in a dose-dependent fashion in PC-3 and VCaP cells (FIGS. 4b&d). Further, stimulation of PC-3 cells with MCP-1 at 100 ng/mL induced p70 S6 kinase phosphorylation but had no effect on GSK3α/β phosphorylation, both of which are down stream targets of Akt (FIG. 4e).

### Effects of MCP-1 on Prostate Cancer Cell Proliferation via Activation of PI3Kinase/Akt

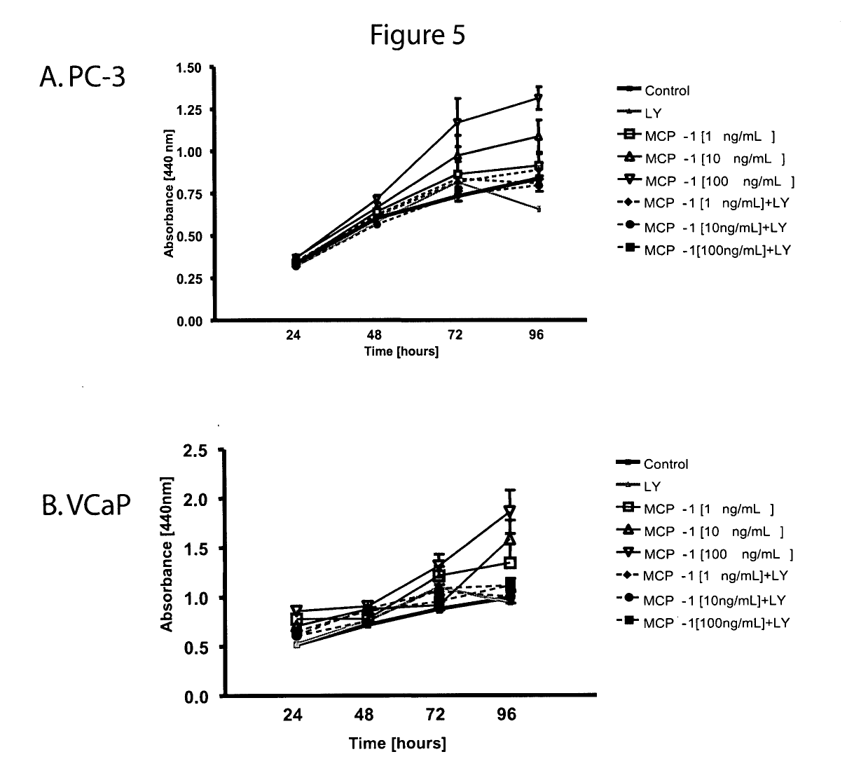

Activation of PI3kinase/Akt is known to be a pro-proliferative signaling pathway (reviewed by, (Song et al., (2005) J Cell Mol Med 9, 59-71)) and previous evidence has shown that MCP-1 stimulates proliferation of macrophages via a PI3kinase/Akt dependent mechanism (Sauvonnet et al., (2002) Mol Microbiol 45, 805-815). To assess the effects of MCP-1 on prostate cancer cell proliferation, PC-3 and VCaP cells were stimulated with increasing concentrations of MCP-1 for 24, 48, 72, and 96 hours in the presence of LY294002 (1 μM), a PI3kinase inhibitor. Both PC-3 and VCaP cells demonstrated enhanced proliferation in response to MCP-1 in a dose-dependent fashion over the 96 hour proliferation assay (solid lines, FIGS. 5a,b). Stimulation of LNCaP cells with MCP-1 had no effect on proliferation compared to the vehicle treated controls (FIG. 5c). The effects of MCP-1 on PC-3 and VCaP cell proliferation were attenuated by the addition of LY294002 (1 μM) during the 96 hour assay (dashed lines, FIGS. 5a,b).

### Differential Expression of MCP-1 Receptors in Prostate Cancer Cell Lines

The differential mRNA expression of CCR2, the high affinity receptor for MCP-1, was quantified by real time PCR and normalized to GAPDH levels expressed in a panel of prostate cancer cell lines. The results are displayed using the Cycle Threshold method previously described (Livak and Schmittgen, (2001) Methods 25, 402-408). CCR2 was variably expressed in RWPE-1, PC-3, VCaP, DU145, LNCaP, C₄₋₂B and DUCaP. PC-3 and VCaP had the highest levels of expression though overall expression of CCR2 in these cell lines was relatively low (Table 1).

### MCP-1 Induces Actin Reorganization in PC-3 Cells

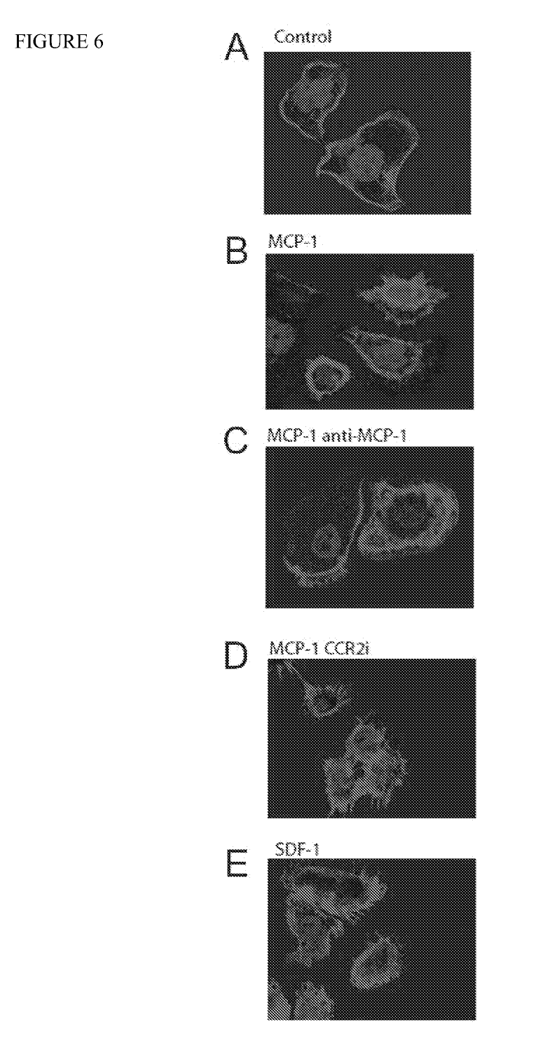

Change in the organization of the actin cytoskeleton is an essential step in the migratory and proliferative phenotype of most cells is known to be linked to G protein coupled receptors (Youngs et al., (1997) Int J Cancer 71, 257-266). CCR2 is a G protein coupled receptor and has been shown to regulate the actin cytoskeleton resulting in a phenotypic change in migration of B cells (Flaishon et al., (2004) Blood 104, 933-941). Additionally, p70 S6 kinase has been shown to regulate actin polymerization and to colocalize with actin at the leading edge during filapodial extensions (Raymond et al., (2002) Neuroscience 109, 531-536). The ability of MCP-1 to stimulate alteration in the actin cytoskeleton in PC-3 cells was assessed. Immunofluorescence revealed increased formation of “finger-like” projections and formation of lamellipodia after 30 min stimulation with MCP-1 (100 ng/mL) compared to control cells (FIG. 6a&b). Further, co-incubation of MCP-1 (100 ng/mL) with an anti-MCP-1 neutralizing antibody prevented lamellipodial formation (FIG. 6c). Inhibition of CCR2b with RS-102895 (1 μM) during MCP-1 stimulation did not prevent actin rearrangement and lamellipodial formation (FIG. 6d).

### In Vivo Imaging of PC-3^{Luc} Cell Metastasis in the Presence of Anti-MCP-1 Antibodies

To visualize the effects of MCP-1 on prostate cancer, an in vivo model of metastasis previously described was utilized (Loberg et al., (2006) Neoplasia 8). PC-3^{Luc} cells were introduced into male SCID mice (n=7) by intracardiac injection and tumor growth was monitored weekly using a CCD camera. At week one post-injection 100% of mice demonstrated at least one focal point of photon emission. Serial bioluminescent images were taken weekly for five weeks. Beginning on Day 14 animals divided into 5 groups and received the following; 1) PBS control, 2) anti-human IgG control, 3) anti-human MCP-1, 4) anti-mouse cVaM control, 5) anti-mouse MCP-1/JE antibodies. Antibodies were given at 2 mg/Kg twice weekly by intraperitoneal injection for three weeks. At Day 35 final images were acquired and the total tumor burden per animal was quantified (FIG. 7a). Overall tumor burden on Day 35 was used to compare efficacy of treatment between treatment groups. Values were normalized to the PBS controls. Administration of anti-human MCP-1 antibodies significantly reduced overall tumor burden by 46.52% compared to the anti-human IgG control antibody (FIG. 7b). Additionally, administration of an anti-mouse MCP-1/JE antibody significantly reduced overall tumor burden by 95.91% compared to the anti-mouse cVaM control antibody (FIG. 7b). There was no difference between the PBS control and the anti-human IgG or the anti-mouse cVaM control antibodies.

### Example 2

### Inhibition of MCP-1 Attenuates Prostate Cancer Epithelial Cell Proliferation and Metastasis In Vivo

Monocyte chemoattractant protein 1 (MCP-1) is a member of the CC chemokine family and is known to promote monocyte chemotaxis. Recent evidence has demonstrated that MCP-1 acts as a potent chemotactic factor regulating stromal—tumor epithelial cells (See Example 1). Using neutralizing antibodies to MCP-1 and the mouse homolog MCP 1/JE, it was demonstrated that treatment of mice with VCaP subcutaneous tumors with both the anti-hMCP-1 (2 mg/Kg; twice weekly by i.p.) and the anti-MCP1/JE (2 mg/Kg; twice weekly by i.p.) antibodies attenuate tumor growth by 42.2% and 55.2% respectively. Treatment with anti-MCP1/JE (2 mg/Kg; twice weekly by i.p.) attenuates PC-3^{Luc} mediated overall tumor burden in an in vivo model of prostate cancer metastasis by 95.9% at 6 weeks post-intracardiac injection. In conclusion, MCP-1 is a potent regulator of prostate cancer motility and proliferation and plays a role in promoting bony metastases.

### Example 3

### MCP-1 TRAP

An MCP-1 TRAP molecule was synthesized by inserting the MCP-1 binding site identified in the high affinity receptor, CCR2, into an Fc fusion vector (PFUSE) to create an Fc fusion protein coupling the binding sequence to the human IgG1 CH2 and CH3 domains of the IgG heavy chain including the hinge region (FIG. 9). Utilizing the Fc fragment will allow the synthesis of a more stable compound with a longer half life in vivo. COS7 cells are transfected with the pFUSE-MCP1 TRAP construct using Lipofectamine 2000 following the manufacturer's instructions. An empty pFUSE vector and a pFUSE-Scrambeld sequence serve as the two negative controls in all experiments. Transfected COS7 cells are selected under Zeocin resistance and clones are isolated and tested for secretion of the MCP1RFc protein by ELISA. Positive subclones producing MCP1 TRAP fusion protein are used for synthesis and purification by protein A-Sepharose affinity chromatography (Pharmacia, Piscataway, N.J.) followed by dialysis against PBS and 0.22 μm filter sterilization. To identify MCP1 TRAP synthesis, the cell culture supernatant is probed by Western blot analysis using an anti-human IgG Fc monoclonal antibody (Chemicon International, Inc.). Purified fusion protein is assessed for binding affinity by ELISA (R & D Systems) by using human recombinant MCP-1 (Chemicon International, Inc.) with increasing concentrations of MCP-1 TRAP.

Recombinant MCP-1 is used to capture the MCP1 TRAP molecule and an anti-human IgG Fc monoclonal antibody (Chemicon International, Inc.) is used as the reporter. Additionally, the ability of MCP-1 TRAP to inhibit MCP-1 mediated migration and proliferation of prostate cancer cells is assessed via migration and proliferation assays in the presence of increasing concentrations of the purified MCP-1 TRAP molecule. Cytotoxicity of the MCP-1 TRAP molecule is determined in vitro by 1) measuring apoptosis via propidium iodide staining and visualization of condensed and fragmented nuclei, and 2) WST-1 cytotoxicity assay (Roche Applied Science, Inc) following the manufacturer's instructions.

Further experiments investigate the effects of inhibition of MCP-1 on prostate cancer growth and metastasis in vivo. In order to determine the toxicity and pharmacokinetics of the MCP-1 TRAP molecule the following studies are performed; 1) Toxicity Studies: MCP-1 TRAP is administered by three i.p. dosing modes: (a) once per week, (b) twice weekly, (c) daily. The MTD (maximum tolerated dose) is defined as the dose that causes a mean 10% body weight loss relative to the saline-treated controls. Each dose is tested in a group of 4-5 mice. The doses of MCP-1 TRAP investigated for toxicity assessment are: bolus: 0 (i.e., saline alone), 2, 20, and 200 mg/kg. Mice are monitored daily for assessment of body weight. In cases in which no weight loss is observed, a value of zero is used for determining the average percent weight loss for the group. In cases in which death occurred, weight loss is defined as the maximal percent weight loss of the expired animal. The MTD is determined by linear interpolation between administered doses. 2) Pharmacokinetics: SCID mice are injected subcutaneously the MTD of MCP-1 TRAP and bleed at 1, 2, 4, 6, 24, 48, 72, 144 hrs post injection. The level of MCP-1 TRAP is measured by ELISA using antibodies that recognize the human Fc region. Additionally, urine and serum MCP-1 levels are measured by ELISA (R&D Systems, Inc.) following the manufacturer's instructions. The maximal serum concentration (Cmax), serum half life (t ½), and area under the curve (AUC=μg×days/mL) of the MCP-1 TRAP molecule is calculated to determine the effective dose and dosage regimen.

To characterize the role of MCP-1 inhibition in attenuated prostate cancer growth and metastases, the of MCP-1 on tumor growth is assessed by implanting PC-3Luc cells subcutaneously in male SCID mice (5-6 weeks) and measuring tumor volume by caliper measurement. Tumor volumes are compared between animals receiving control of the MTD of MCP-1 TRAP twice/week by i.p. injection. Tumor volumes are monitored and calculated. Identification of neovascularization and macrophage infiltration are accomplished histologically. To assess the role of MCP-1-regulated metastasis of prostate cancer cells in vivo, the PC-3Luc cell line expressing the bioluminescent-catalyzing enzyme luciferase construct as previously described by our laboratory is utilized (Rice et al., J Biomed Opt, 2001. 6(4): p. 432-40; Kalikin et al., Cancer Biol Ther, 2003. 2(6): p. 656-60). One week after intracardiac injection of PC-3Luc cells (200,000 cells in 100 μL DPBS) into intact male SCID mice a subgroup of mice receive a MCP-1 TRAP. A control Fc fusion protein with a non-specific sequence serves as a control to the MCP-1 TRAP. To assess metastasis anesthetized animals are injected with luciferin intraperitoneally (100 μL at 40 mg/mL) and imaged using a charge-coupled device (CCD) system at The University of Michigan In Vivo Cellular and Molecular Imaging Center. To detect a 25% difference with a power of 0.9 and confidence interval of 95% CI in metastatic rate between control and anti-MCP-1 therapy requires 10 animals in each arm. As evident by distinct bioluminescent foci, sites of micrometastases are detected, identified and quantified as previously described (Kaliken et al., supra). Histoligical analysis is performed by a pathologist after necropsy.

### Example 4

### Combination Therapy

This Example describes combination therapies for prostate cancer and prosate cancer metastasis.

### A. Methods

Description of CNTO888 and C1142 and Control Antibodies

CNTO888 is a human IgG1κ antibody that neutralizes human CCL2. C1142 is a rat/mouse chimeric antibody that neutralizes mouse CCL2/JE. Clinical grade human IgG (huIgG) served as a negative control for CNTO888, while C1322 rat/mouse chimeric nonspecific antibody (Centocor) served as a negative control for C1142.

Cell Culture

PC-3Luc prostate cancer cell lines were generated as previously described (Loberg et al., 2006. Neoplasia. 8:69-78) and maintained in RPMI 1640+10% Fetal Calf Serum (FCS) (Invitrogen Corp., Carlsbad, Calif.). Cells were passaged by trypsinization using 1× Trypsin+EDTA (Invitrogen Corp.), resuspended in appropriate growth media, and were used within 10 passages of each other for consistency.

Proliferation Assay

Cells were seeded at a density of 1×10⁵ cells/ml for PC-3Luc cells in a 96 well plate in RPMI+10% FBS (FCS is mentioned in previous section). Twenty-four hours after seeding, media was changed to either serum-free RPMI or RPMI+10% FBS. Cell growth was determined 72 hours later using the WST-1 assay (Pierce Biotech, Inc.) following the manufacturer's instructions.

Migration Assay

Increasing concentrations of CCL2 (1-100 ng/mL) or conditioned media was added to the lower chamber of a 24-well plate. Cells were harvested by EDTA release and resuspended in serum free media at 5×10⁴ cells/ml. 2.5×10⁴ cells were added to the upper chamber of the transwell insert and incubated for 24 hours at 37° C. and 5% CO² atmosphere. At the end of the incubation period, the cells were fixed with 4% formaldehyde in PBS for 5 minutes. Non-adherent cells were removed from inside the inserts with a cotton-tipped applicator. Cells which had migrated to the underside of the insert were stained with 0.5% crystal violet for 5 minutes and rinsed thoroughly with tap water. Inserts were allowed to dry and the cells were counted using an inverted microscope.

Immunoblot Analysis

Cells were lysed in RIPA buffer (50 mM Tris-HCl, pH 7.4, 1% NP-40, 150 mM NaCl, 1 mM EDTA, 1 mM PMSF, 1 mM Na₃VO4, 1 mM NaF, 1 μM okadaic acid and 1 μg/mL aprotinin, leupeptin and pepstatin). Proteins were separated under reducing conditions by SDS-PAGE and transferred onto nitrocellulose membrane. The membranes were blocked with 5% milk in TBST (0.1% Tween in TBS) for 1 hour at room temperature, then they were incubated overnight at 4° C. with primary antibodies. Membranes were washed 3 times prior to incubation with HRP-conjugated secondary antibodies (Cell Signaling, Beverly, Mass.) for 1 hour at room temperature. Protein expression was visualized by ECL chemiluminescence (Promega, Madison, Wis.) and quantitated using Image J software (NCl, Bethesda, Md.).

Tissue Microarray (TMA) Analysis

TMAs were manufactured as previously described (Yoshimura et al., 1989. J. Immunol. 142:1956-1962). Briefly, needle cores were retrieved from tissue specimens in paraffin blocks. Tissue cores of 0.6 mm in diameter were arrayed vertically in triplicate in a new paraffin block. Array slides were stained with immunoperoxidase stains using anti-CCR2 (Abcam, Inc., Cambridge, Mass.) and the DAKO AutoStainer and EnVision+Peroxidase development kits from DAKO Cytomation (Carpinteria, Calif.). Microwave antigen retrieval was performed in a citrate buffer (pH 6.0) for 10 minutes on all slides. Arrays were analyzed by a pathologist and percentage and intensity of epithelial cells stained were recorded. Staining intensity was ranked. Data is presented as mean +/−standard error.

Bioluminescent In Vivo Model of Metastatsis

Bioluminescent imaging of PC-3Luc was preformed as previously described through The University of Michigan Small Animal Imaging Resource facility (MSAIR) (Loberg et al., 2006. Neoplasia. 8:578-586). Briefly, PC-3Luc cells were introduced into male SCID mice (5-6 wks of age) by intracardiac injections. Mice were serially imaged weekly for up to 12 weeks using a CCD IVIS system using a 50 mm lens (Xenogen Corp, Alameda, Calif.) and the results were analyzed using LIVING IMAGE software (Xenogen Corp.). Mice were separated into groups and treatment began at week 2 post-intracardiac injection and mice received 2 mg/Kg antibody twice weekly by intraperitoneal (i.p.) injection (for up to eight weeks) and/or 40 mg/Kg Taxotere i.p. injection once per week for three weeks. Mice were injected with luciferin (40 mg/mL) intraperitoneally and ventral images were acquired 15 min postinjection under 1.75% isofluorane/air anesthesia. Total tumor burden of each animal was calculated using regions of interest (ROIs) that encompassed the entire animal. Animals were sacrificed after week 6 image and individual organs were harvested and immediately placed in formalin.

Histology

Animals were sacrificed by cervical dislocation and tissue specimens were harvested and fixed in formalin for hematoxylin and eosin histological analysis following routine protocols. Soft tissue specimens were prepared for IHC by placing 5 μm sections were on charged glass slides and stained while tibias were decalcified in Cal-Ex II (Fisher Scientific) decalcifying solution for 24-48 hours and 5 μm sections were placed on charged glass slides.

Statistics

Data was analyzed with GraphPad Prizm software. A One-way-ANOVA analysis was used with Bonferroni's post-hoc analysis for comparison between multiple groups. A Students T-test was used for comparison between two groups. Significance was defined as a p value <0.05.

### B. Results

CNTO888 Inhibited PC-3Luc Cell Proliferation and Migration In Vitro

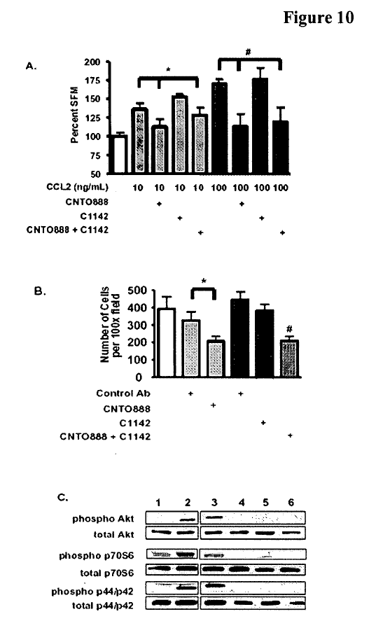

To determine the potential of inhibiting CCL2 and the role CCL2 inhibition would play on prostate cancer cells, PC-3 cells were stimulated in vitro with hrCCL2 (10-100 ng/mL) for 72 hours in the presence of an anti-human CCL2 neutralizing antibody (CNTO888) or/and anti-mouse CCL2/JE neutralizing antibody (C1142) (FIG. 10a). An increase in cell viability, as a measure of proliferation, was determined using the WST-1 method. The data revealed an inhibition of hrCCL2-induced proliferation by CNTO888 compared to the human IgG control antibody and the anti-CCL2/JE (C1142) antibody [CCL2 (10 ng/mL): 136.2±7.81; CNTO888: 112.52±10.2; C1142: 132.18±3.48; CNTO888+C1142: 108.22±9.89, mean ±SD] and [CCL2 (100 ng/mL): 168.15±5.44; CNTO888: 132.93±17.08; C1142: 165.92±15.22; CNTO888±C1142: 118.93±19.29, mean ±SD]. Similarly, CNTO888 attenuated hrCCL2-induced migration compared to either control or C1142 antibodies [Control: 393±67; CNTO888: 217±29; C1142: 371±36; CNTO888±C1142: 209±24, mean ±SD] (FIG. 10b). Previously, an upregulation of Akt activity in PC-3 cells stimulated with hrCCL2 was reported (Loberg et al., 2006. Neoplasia. 8:69-78). The presence of CNTO888 attenuated Akt, p70 S6 kinase and p44/p42 MAPK activation in response to hrCCL2 stimulation (FIG. 10c). These results indicated that prostate cancer cells responded to CCL2 by enhanced proliferation and migration, and that an anti-CCL2 antibody could inhibit these activities.

CCR2 Expression Correlates with Prostate Cancer Progression and Metastasis

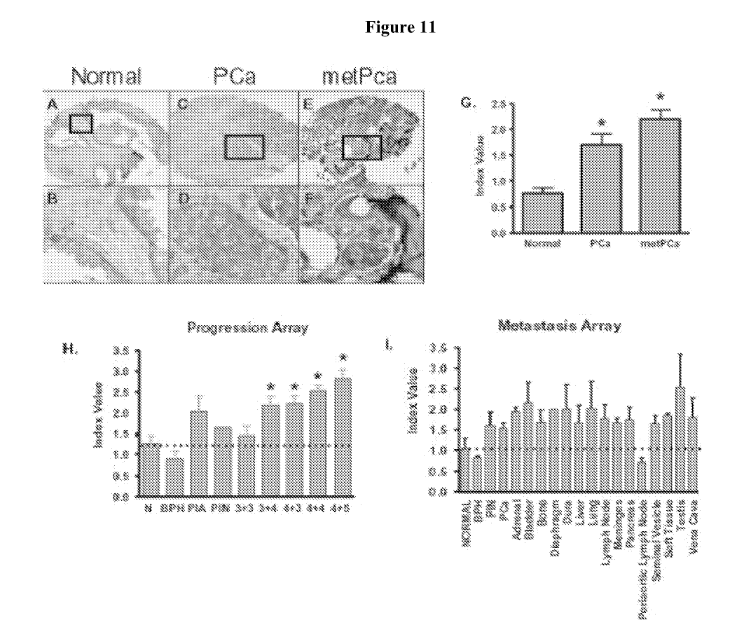

To determine the clinical importance of targeting the CCL2/CCR2 signaling pathway in prostate cancer, CCR2 receptor expression on prostate epithelial cells was analyzed by tissue microarray (TMA) in collaboration with the TMA core at the University of Michigan (FIG. 11). Prostate cancer epithelial cell CCR2 expression demonstrated a correlation with PIA (proliferative inflammatory atrophy) and Gleason score with significantly elevated levels of expression in Gleason >7 tumors (FIG. 11h). TMA analysis revealed a significant increase in CCR2 expression in metastatic tissue compared to primary prostate cancer, though no significant difference in CCR2 expression was observed when soft tissue metastases were compared to bone metastases (FIGS. 11g, i).

Anti-CCL2 Antibodies Decrease Tumor Burden In Vivo

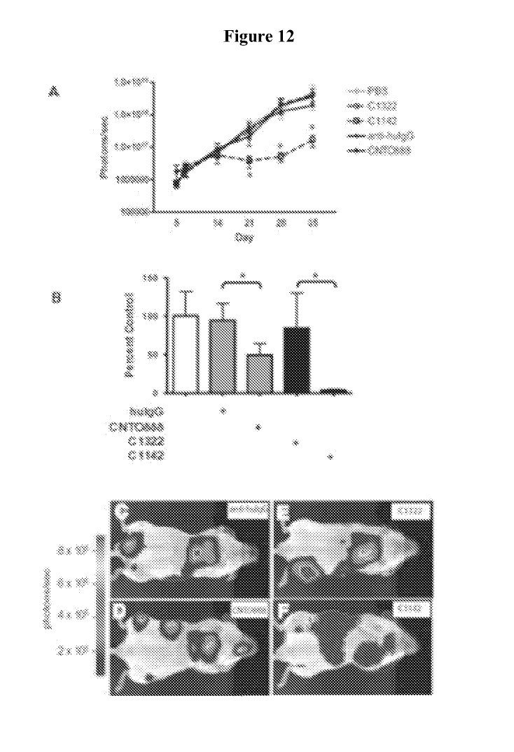

To visualize the effects of CCL2 inhibition on prostate cancer, an in vivo model of metastasis was utilized as previously described (Loberg et al., 2006. Neoplasia. 8:578-586). PC-3Luc cells were introduced into male SCID mice (n=7) by intracardiac injection and tumor growth was monitored weekly. At week one post-injection, 100% of mice demonstrated at least one focal point of photon emission. Serial bioluminescent images were taken weekly for five weeks. Beginning on Day 14, animals were divided into 5 groups and received the following treatment/antibodies: 1) PBS control, 2) huIgG control, 3) anti-human CCL2 CNTO888, 4) mouse control antibody (C1322), and 5) anti-mouse CCL2/JE (C1142). Antibodies were given at 2 mg/Kg twice weekly i.p. for three weeks. At Day 35, total tumor burden per animal was quantified and efficacy between treatment groups ascertained (FIG. 12a).

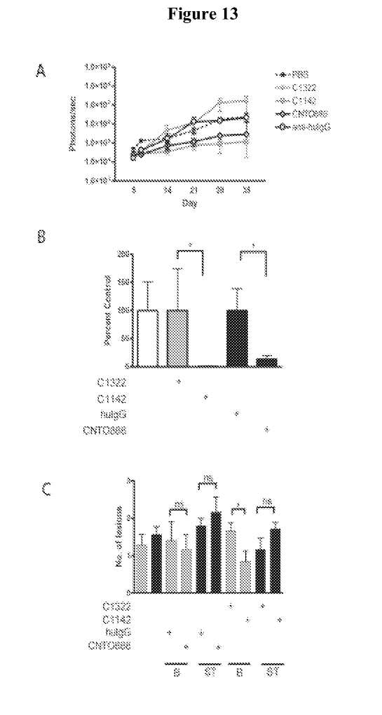

Administration of anti-human CCL2 antibody (CNTO888) significantly reduced overall tumor burden by 46.52% compared to the anti-human IgG control antibody; FIGS. 12b, c&d). Additionally, administration of an anti-mouse CCL2/JE antibody (C1142) significantly reduced overall tumor burden by 95.91% compared to the mouse control antibody (FIGS. 12b, e&f). There was no difference between the PBS control and the huIgG or the mouse control antibodies. Comparison of tumor burden between treatment groups in a specific bone site (the tibia) revealed similar significant inhibition of tumor growth (FIG. 13).

As prostate cancer metastases present predominantly as bone lesions, tumor burden localized in the tibia of the intracardiac injected mice described above was analyzed. The tibia is a common site of metastasis for PC-3Luc cells as described previously (Kalikin et al., 2003. Cancer Biol Ther. 2:656-660). Tibia specific tumor burden was analyzed for five weeks post-intracardiac injection (FIG. 13a).

Tumor burden on Day 35 was used to compare efficacy of treatment between treatment groups as stated above. Administration of anti-human CCL2 antibody significantly reduced tibia-specific tumor burden by 45.49% for the anti-human antibody CNTO888 (FIG. 13b) and compared to the huIgG control antibody (FIG. 13b).

Additionally, administration of an anti-mouse CCL2/JE antibody significantly reduced tibia-specific tumor burden by 98.66% for the anti-mouse antibody (FIG. 13b). Further, administration of the anti-mouse CCL2/JE (C1142) significantly reduced the number of bone-specific metastatic lesions as identified by visual confirmation of luciferase signal upon imaging (FIG. 13c). These results indicate that inhibition of either the host stromal-derived mouse CCL2 or the tumor-derived human CCL2 can attenuate the formation of bone metastases, suggesting that each may play a role in this process.

Single Agent Anti-CCL2 Compared to Single Agent Docetaxel In Vivo

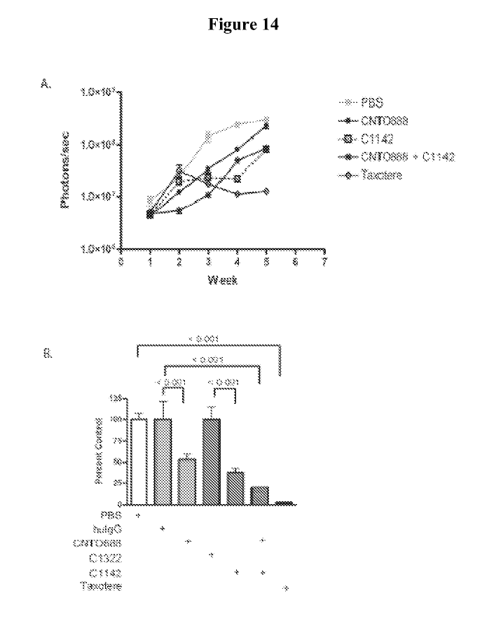

To further determine the efficacy of CCL2 inhibition in advanced prostate cancer, CNTO888 and C1142 as single agents were compared to single agent docetaxel at a maximally tolerated dose (MTD) that was previously established (FIG. 14a). CNTO888 (2 mg/Kg, i.p. twice weekly) demonstrated a significant reduction in tumor burden by Day 35 (52.78% of PBS-treated animals) while C1142 (2 mg/Kg, i.p. twice weekly) and C1142+CNTO888 (2 mg/Kg each, i.p. twice weekly) resulted in a greater decrease in total tumor burden (38.07% and 19.83% of PBS-treated animals, respectively) compared to CNTO888 alone. Neither CNTO888, C1142, nor CNTO888+C1142 was as effective as a single agent when compared to single agent docetaxel (40 mg/Kg, i.p. q3 wks) (3.19% of PBS-treated animals at Day 35) (FIG. 14b).

Anti-CCL2Antibodies in Combination with Docetaxel Induce Tumor Regression In Vivo

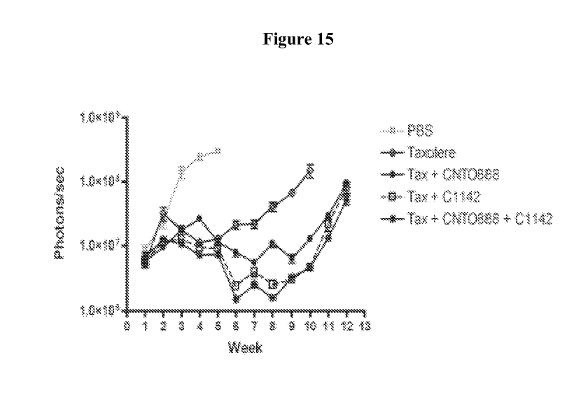

To further determine the efficacy of combination therapy with docetaxel and CCL2 inhibition in advanced prostate cancer, CNTO888 and C1142 in combination with docetaxel were compared to single agent docetaxel (FIG. 15). Treatment was initiated on week 2 post-intracardiac injection, and mice received either single agent docetaxel (MTD—40 mg/Kg, i.p. qw for 3 weeks) or docetaxel in combination with anti-CCL2 antibodies (2 mg/Kg, i.p. twice weekly). Docetaxel treatment was stopped after 3 weeks and animals were maintained on antibodies for an additional 3 weeks (until week 8) after which all treatment was stopped and tumor burden was monitored. Mice treated with docetaxel alone displayed a decrease in tumor burden while receiving therapy, but once the treatment stopped the mice began to develop additional tumor burden. Mice treated with a combination of docetaxel and anti-CCL2 antibodies demonstrated a significant regression of tumor burden compared to animals receiving single agent docetaxel (FIG. 15, Table 2). Furthermore, continued administration of anti-CCL2 antibodies after treatment with docetaxel was discontinued showed that the mice maintained the decreased tumor burden compared to mice that did not receive antibody therapy (FIG. 15, Table 2). Once antibody treatment was discontinued, tumor burden began to increase again until the mice were euthanized at week 12. These results demonstrate that the combination of docetaxel and anti-CCL2 antibodies was more efficacious than docetaxel alone, and that the antibody therapy played a role in the maintenance of tumor regression.

TABLE 2

| No. Animals | | | | |
|---|---|---|---|---|
| per Group | Treatment | Wk 6 | Wk 9 | Wk 11 |
| 10 | PBS | 1 of 10 | | |
| 10 | hulgG | 2 of 10 | | |
| 9 | C1322 | 0 of 9 | | |
| 9 | CNTO888 | 1 of 9 | | |
| 9 | C1142 | 0 of 9 | | |
| 9 | CNTO888 + C1142 | 0 of 9 | | |
| 9 | Taxotere | 2 of 9 | 1 of 9 | |
| 9 | Taxotere + CNTO888 | 6 of 9 | 3 of 9 | 2 of 9 |
| 9 | Taxotere + C1142 | 7 of 9 | 4 of 9 | 3 of 9 |
| 9 | Taxotere + CNTO888 + C1142 | 9 of 9 | 6 of 9 | 3 of 9 |

Wk 6 = First week after cessation of Taxotere
Wk 9 = First week after cessation of Antibodies

### Example 5

### CCL2 Mediates Tumor Establishment in the Bone Microenvironment

This example further describes the role of CCL2 (MCP-1) in prostate cancer metastasis to the bone.

### A. Methods

### Materials

Human Parathyroid hormone related protein (PTHrP)-(1-34) was from Bachem (Torrance, Calif.). Collagenase A was from Roche Biomedicals, and trypsin was obtained from Life Technologies (Gaithersburg, Md.).

### Cell Culture

VCaP cells were obtained from a lumbar vertebral metastatic lesion through the Rapid Autopsy program at the University of Michigan (Korenchuk et al., 2001; 2: 163-168; Loberg et al., Urol. One. 2006; 24:161-168). Cells were cultured in Dulbecco's Modified Eagle Medium supplemented with 10% Fetal Bovine Serum and 1% antibiotic/antimicotic and incubated at 37° C. PC3 prostate cancer cells were obtained from the American Type Culture Collection (Manassas, Va.). Cells were cultured in RPMI-1640 with L-Glutamine (Cambrex Bioproducts) and supplemented with 10% Fetal Bovine Serum and 1% antibiotic/antimicotic. Cells were incubated at 37° C. and subcultured according to ATCC specifications. MC3T3-E1 subclone 4 cells (MC-4) with high osteoblast differentiation potential were maintained and passaged every 4-5 days as previously described. Briefly, cells were grown in AMEM (Gibco) containing 100 units/ml penicillin and streptomycin and 10% FBS. MC3T3 cells were plated at 40,000-50,000 cells/cm2, and differentiation was induced with the addition of ascorbic acid (50 μg/ml) for 7 days. The culture medium was changed at days 1, 3, 5, and 7. Cells were subsequently treated with vehicle or PTHrP at 10 nM for indicated time.

Primary mouse calvarial cells were isolated as previously described. Briefly, calvaria of newborn mice at day 4 were dissected, isolated, and subjected to sequential digestions in collagenase A (2 mg/ml) and 0.25% trypsin for 20, 40, and 90 minutes. Cells from the third digest were washed, counted, and plated in αMEM with 10% FBS containing 100 U/ml of penicillin and streptomycin. Primary cultures were used without passage. For differentiation, both MC-4 and primary cells were induced to differentiate and form mineralized matrix with the addition of ascorbic acid (50 μg/ml) and β-glycerophosphate (10 mM) with media replacement (every 2 days). In proliferation phase, MC-4 cells were serum staved for 24 h before addition of PTHrP or vehicle for indicated time. Otherwise, cells were treated with PTHrP or vehicle for indicated time without serum starvation.

### CCL2 ELISA

A mouse CCL2 specific ELISA kit was purchased from BD Biosciences (San Diego, Calif.) to measure the protein level of CCL2 in cell culture media. Assays were performed as recommended by the manufacturer to determine levels of secreted CCL2 in MC-4 or primary cell culture media. Values were calculated from standard curves set up for each assay. The data are based on a triplicate experiments performed independently. Data are shown as mean +SEM in the figures.

### Intratibial Injection of VCaP and PC3 Prostate Cancer Cells

Five week old CB 17 severe combined immunodeficient mice were obtained from Charles River Laboratories. VCaP and PC3 cells were cultured in T-75 flasks to 100% confluence. Cells were trypsinized, washed and counted using a hemocytometer counting chamber. 1.0×10⁷ Cells were resuspended in 200 μL of sterile phosphate buffered saline and placed on ice prior to injection. Mice were sedated with 1.7% isofluorane mixed with air five minutes prior to injection. During the procedure mice were kept sedated using nose cone delivered 1.7% isofluorane and air. A 27 gauge needle was used to bore a hole into the marrow cavity through the tibial plateau, into the left tibia of the mouse. The 27 gauge needle was removed and 10 μL of cells (5×10⁵ total cells) were injected into the marrow cavity using a 28 gauge Hamilton Syringe. Mice were closely monitored twice weekly. After six weeks the mice were x-rayed once weekly using in order to qualify and quantify tumor growth. After 8 weeks animals were sacrificed. Tibias were removed and placed in 10% formaldehyde for exactly 24 hours. After 24 hours the bones were transferred to 70% Ethanol. Bone mineral density was characterized both peripheral duel-energy x-ray absorptiometry and x-ray microcomputed tomography.

### Treatment with Anti-CCL2/JE (C1142)

Prior to intratibial injection, mice were pre-treated with 2 mg/Kg anti-CCL2/JE (C1142). Control mice were treated with either 2 mg/Kg isotype control antibody (C1322) or PBS. Following intratibial injection with PC-3 or VCaP prostate cancer cells, mice continued treatment twice weekly until the end of the experiment.

### Histology

Xenograft tumors were harvested and placed in fresh 10% formalin. Tibias were decalcified for X days prior to paraffin embedding. Paraffin embedded specimens were section into 5 μm sections and placed on glass slides. Hematoxylin and eosin stain was performed per the manufacturer's instructions (Sigma, Inc.).

### Serum TRAP5b Activity

Serum TRACP5b activity was measured by ELISA (Immunodiagnostic Systems, Inc.) following the manufacturer's instructions.

### B. Results

### Co-Culture of HBME Cells with Prostate Cancer Cell Conditioned Media Show an Increase in CCL2 Expression by HBME Cells.

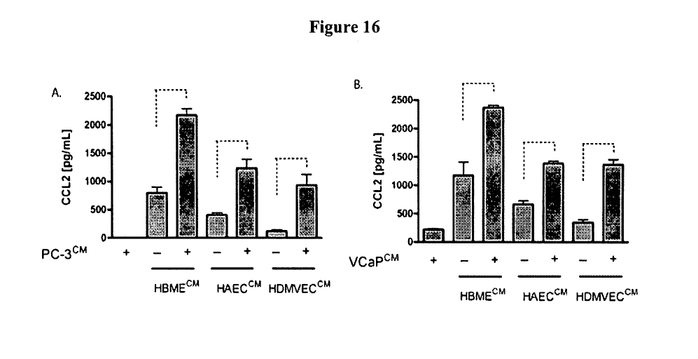

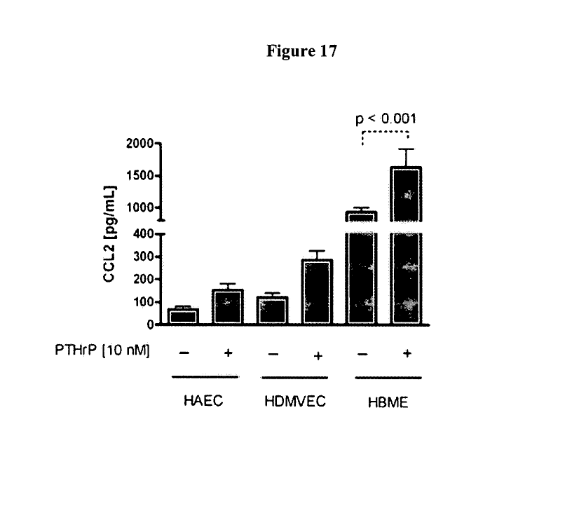

To determine the role of CCL2 in the development of prostate cancer-bone metastasis the level of CCL2 was analyzed by ELISA. Previously it was demonstrated that CCL2 is expressed at high levels by bone marrow endothelial cells (Loberg et al., 2006. Neoplasia. 8:578-586). Here CCL2 levels were measured in vitro when endothelial cells were cultured with prostate cancer cell conditioned media. Incubation with PC-3 and VcaP conditioned media significantly increased the levels of CCL2 expression by all endothelial cells analyzed (bone marrow endothelial cells—HBME, aortic endothelial cells—HAEC, and dermal microvascular endothelial cells—HDMVEC) (FIG. 16). HBME cells cultured in either PC-3 or VCaP conditioned media secreted significantly higher levels of CCL2 compared to HAEC or HDMVEC cultured in similar conditioned media (FIG. 16). Further, stimulation of HBME, HAEC and HDVMEC cells with PTHrP (10 nM) increased their respective synthesis of CCL2 (FIG. 17).

### Osteoblasts Secrete CCL2 in Response to PTHrp

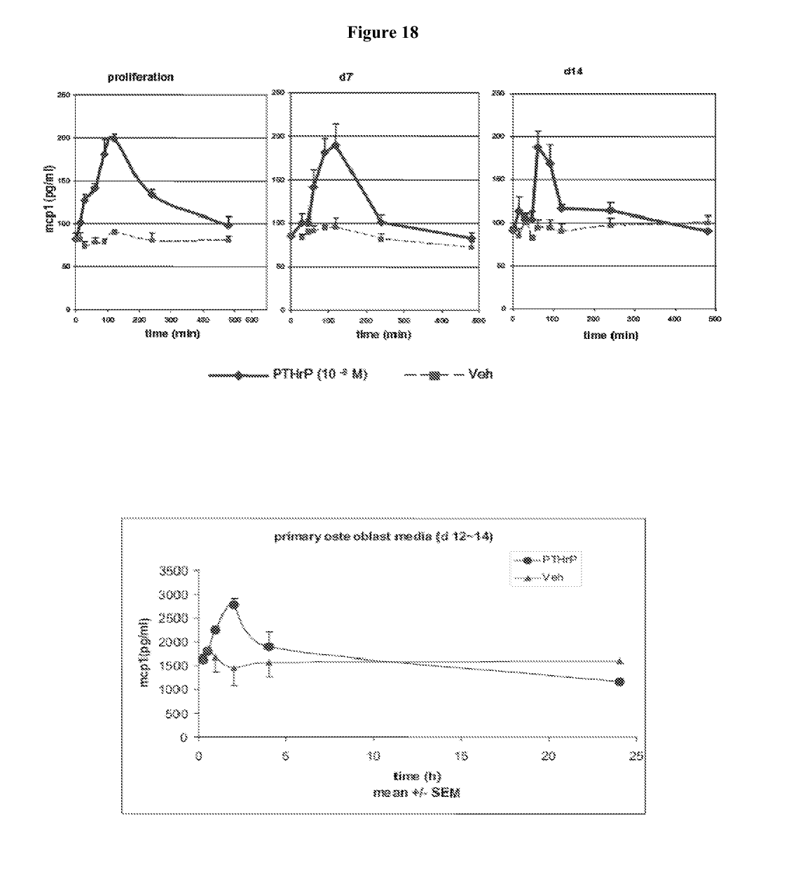

Osteoblasts (OB) have previously been reported to secrete CCL2 and PTHrP is known to be an important stimulator of osteoblast activity in prostate cancer. To test the hypothesis that PTHrP stimulated OBs to secrete elevated levels of CCL2 contributing to the favorable microenvironment in the bone marrow compartment for prostate cancer metastasis, MC3T3-E1 subclone 4 (MC-4) OB cells were stimulated with PTHrP (10 n-M) in vitro. Stimulation of MC-4 cells induced CCL2 expression rapidly increased within 2 hours of stimulation with PTHrp followed by a subsequent return to basal levels. The increase in CCL2 in response to PTHrP was observed in MC-4 cells stimulated at day 7 and day 14 after induced-differentiation by ascorbic acid treatment. Additionally, primary mouse calvaria osteoblasts increased CCL2 expression when stimulated with PTHrP and the maximal CCL2 response was observed similarly at 2 hours post-stimulation with PTHrP (FIG. 18).

### Xenograft Implantation and Collection of Bone Marrow after 4 Weeks Induced Elevation of CCL2 in the Bone Marrow Compartment Prior to Tumor Metastasis.

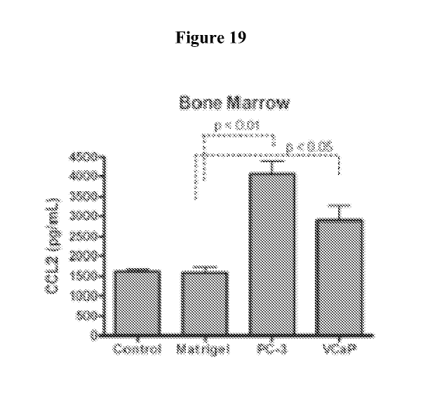

To determine the ability of a primary tumor to alter the bone marrow microenvironment to support metastases prior to the metastatic event PC-3 and VCaP cell xenografts were implanted subcutaneously and allowed to develop for 4 weeks. At 4 weeks bone marrow aspirates were collected from the tibias and analyzed for CCL2 expression. The presence of PC-3 and VcaP cell xenografts significantly increased the levels of CCL2 expression in the bone marrow compared to control animals (Control: 1623±119.0, Matrigel: 1596±253.3, PC-3: 4056±593.7, VCaP: 2901±648.8 [pg/mL]; mean ±SD) (FIG. 19).

### Intratibial Injection in Mice Pre-Treated with C1142

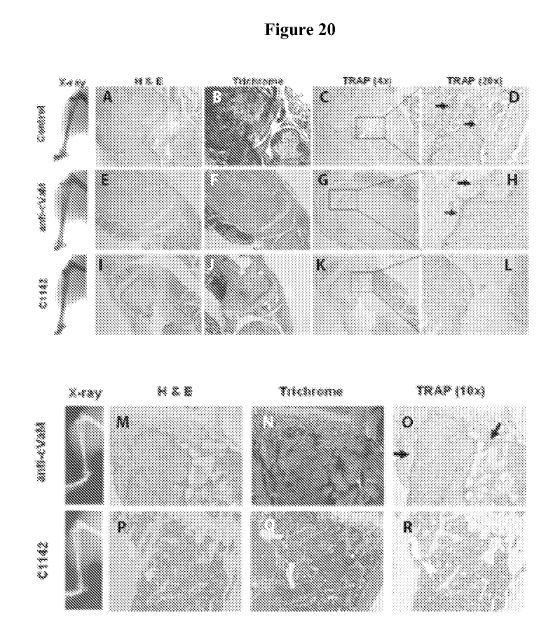

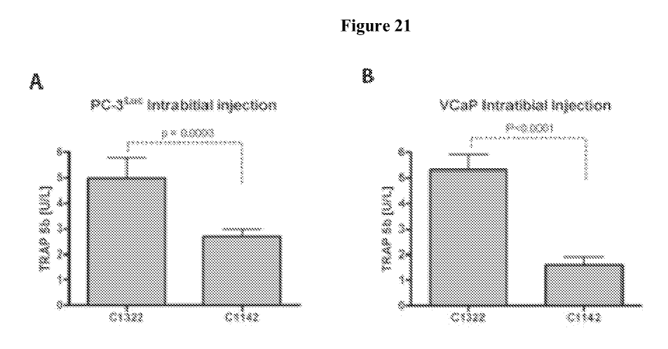

Intratibial injection in mice pre-treated with C1142 demonstrated 1) decreased tumor burden, 2) maintenance of bone volume, 3) decrease in osteoclast activity by TRAP staining. To determine the role of CCL2 in prostate cancer bone establishment and growth, systemic inhibition of CCL2 was accomplished using neutralizing antibodies that target mouse CCL2/JE (C1142). Mice were inoculated with either PC-3 or VCaP prostate cancer cells. PC-3 bone lesions have been well characterized and establish osteolytic bone lesions. VCaP cells were originally isolated from a vertebral metastatic lesion during from an autopsy as part of the University of Michigan Rapid Autopsy Program. PC-3 cells are androgen receptor negative and are androgen independent while VCaP cells express a wild type androgen receptor and are androgen sensitive. Both PC-3 and VCaP cells are known to express the CCL2 receptor, CCR2 (Loberg, 2006, supra). Here mice were injected with PC-3 or VCaP by intratibial injection and bone lesions were monitored by weekly radiological imaging. PC-3 cells established visible osteolytic lesions by week 6 and VCaP cell establish a mixed osteolytic/osteoblastic lesion by week 10. Tibias were harvested and examined by immunohistochemical analysis (FIG. 20). Overall tumor burden was significantly decreased in tibias of mice receiving C1142 (anti-CCL2/JE) antibodies compared to control antibodies (C1322). Bone destruction was decreased, as measured by trichrome staining for mineralized bone content, in animals receiving C1142. Additionally, inhibition of CCL2 attenuated TRAP+ osteoclast staining suggesting a decrease/inhibition of osteoclast activity. Mice inoculated with VCaP cells failed to develop tibial lesions when treated with anti-CCL2 antibodies. Analysis of serum markers of osteoclast activity revealed a significant decrease in TRAP5b serum concentration in animals receiving anti-CCL2 antibodies compared to controls (PC-3 control C1322: 4.975±0.7926, C1142: 2.709±0.284) (VCaP control C1322: 5.3.14±0.6033, C1142: 1.612±0.32) (FIG. 21).

### Example 6

### CCL2 Regulates Macrophage Infiltration

This example further describes the role of CCL2 (MCP-1) in prostate cancer metastasis to the bone.

### A. Methods

### Materials

Human recombinant CCL2 was obtained from Chemicon International (Temecula, Calif.), anti-phospho AktSer473, anti-Akt, anti-phospho p44/p42, and anti-total p44/p42 were obtained from Cell Signaling (Beverly, Mass.), and all other reagents were obtained from Sigma-Aldrich.

### Description of CNTO888 and C1142 and Control Antibodies

CNTO888 is a human IgG1κ antibody that neutralizes human CCL2. C1142 is a rat/mouse chimeric antibody that neutralizes mouse CCL2/JE. CNTO888 and C1142 do not cross-react with or neutralize mouse CCL2/JE or human CCL2, respectively. Clinical grade human IgG (huIgG) served as a negative control for CNTO888, while C1322 rat/mouse chimeric nonspecific antibody (Centocor) served as a negative control for C1142.

### Cell Culture

VCaP cells are a human prostate cancer cell line derived from a vertebral bone metastasis. VCaP cells were maintained in DMEM 1640+10% Fetal Calf Serum (FCS) (Invitrogen, Carlsbad, Calif.). Cells were passaged by trypsinization using 1× Trypsin+EDTA (Invitrogen) and resuspended in appropriate growth media.

### Xenograft Model of Tumorigenesis

Xenograft tumors were established as previously described (Loberg et al., Urol Oncol 24 (2006) 161-168). Briefly, male SCID mice (5-6 weeks of age) were injected subcutaneously in the flank with 1×10⁶ VCaP cells in 200 μL Matrigel (BD Biosciences, Inc.). Tumor volumes were calculated by caliper measurement performed weekly to monitor and track tumor growth (tumor volume=L×W×W×0.56). Mice were separated into one of four groups: 1) huIgG, 2) C1322 control mouse antibody, 3) anti-CCL2 (CNTO888), 4) anti-CCL2/JE (C1142). Mice were treated with 2 mg/Kg antibody twice weekly by intraperitoneal injection, beginning on Day 28 and for the remainder of the study.

### Histology

Xenograft tumors were harvested and placed in fresh 10% formalin. Tumors were paraffin-embedded and 5 μm sections were cut and placed on glass slides. Hematoxylin and eosin stain was performed per the manufacturer's instructions (Sigma, Inc.). Identification of neovascularization was accomplished by labeling with an anti-CD31 antibody and macrophage infiltration was identified using an anti-CD68 antibody. Tissue sections were incubated for 10 minutes in citrate buffer, pH 6.0 and microwaved. Sections were incubated with anti-CD31 (DakoCytomation, Inc.; 1:50) or anti-CD68 (DaKoCytomation, Inc.; 1:1600) for 30 minutes and detected with LSAB+detection/DAB (3,3′-Diaminobenzidine; Sigma, Inc.) for 5 minutes. Slides were dipped in hematoxylin for 1 second as a counterstain.

### Endothelial Tube Formation Assay

In vitro tube formation was performed as previously described (Zhou et al., Int J Cancer 110 (2004) 800-806). Growth factor reduced MATRIGEL was diluted with cold serum-free medium to a concentration of 10 mg/ml. 50 μl of the solution was added to each well of a 96-well plate and allowed to form a gel at 37° C. for 30 min. HDMVEC (human dermal microvacular endothelial cells) cells (150,000 cells/ml) in VCaP conditioned media (VCaP CM) were added to each well and incubated overnight at 37° C. in 5% CO₂. Either control antibodies (hulgG or C1322; 30 μg/mL) or anti-CCL2 antibodies (CNTO888 and/or C1142; 30 μg/mL) were added to the conditioned media. Under these conditions, EC will form delicate networks of tubes that are detectable within 2-3 h and are fully developed after 8-12 h. After overnight incubation the wells were washed, and the MATRIGEL and its endothelial tubes were fixed with 3% paraformaldehyde. Tube formation was quantified by counting the number of sprouts that developed per objective field (100×) and assays were performed in triplicate from 3 independent experiments.

### Macrophage Migration

Human recombinant CCL2 was used as the chemoattractant in the lower chamber of a modified Boyden chamber. Cells were harvested by 0.5 μM EDTA release and resuspended in serum free media at 5×10⁴ cells/ml. 2.5×10⁴ cells were added to the upper chamber of the transwell insert and incubated for 24 hours at 37° C. and 5% CO₂ atmosphere. At the end of the incubation period, the cells were fixed with 4% formaldehyde in PBS for 5 minutes. Non-adherent cells were removed from inside the inserts with cotton-tipped swabs. Cells that had migrated to the underside of the insert were stained with 0.5% crystal violet for 5 minutes and rinsed thoroughly with tap water. Inserts were allowed to dry and the cells were counted using an inverted microscope.

### Immunoblot Analysis

Cells were lysed in RIPA buffer (50 mM Tris-HCl, pH 7.4, 1% NP-40, 150 mM NaCl, 1 mM EDTA, 1 mM PMSF, 1 mM Na₃VO₄, 1 mM NaF, 1 μM okadaic acid and 1 μg/mL aprotinin, leupeptin and pepstatin). Proteins were separated under reducing conditions by SDS-PAGE and transferred onto nitrocellulose membranes. The membranes were blocked with 5% milk in TBST (0.1% Tween in TBS) for 1 hour at room temperature, then were incubated overnight at 4° C. with primary antibodies. Membranes were washed 3 times prior to incubation with HRP-conjugated secondary antibodies (Cell Signaling) for 1 hour at room temperature. Protein expression was visualized by ECL chemiluminescence (Promega, Madison, Wis.) and quantitated using Image J software (NCl, Bethesda, Md.).

### Statistics

Data was analyzed with GraphPad Prism software. A One-way-ANOVA analysis was used with Bonferroni's post-hoc analysis for comparison between multiple groups. A Students T-test was used for comparison between two groups. Significance was defined as a p value <0.05.

### B. Results

### Xenograft Model of Tumor Growth

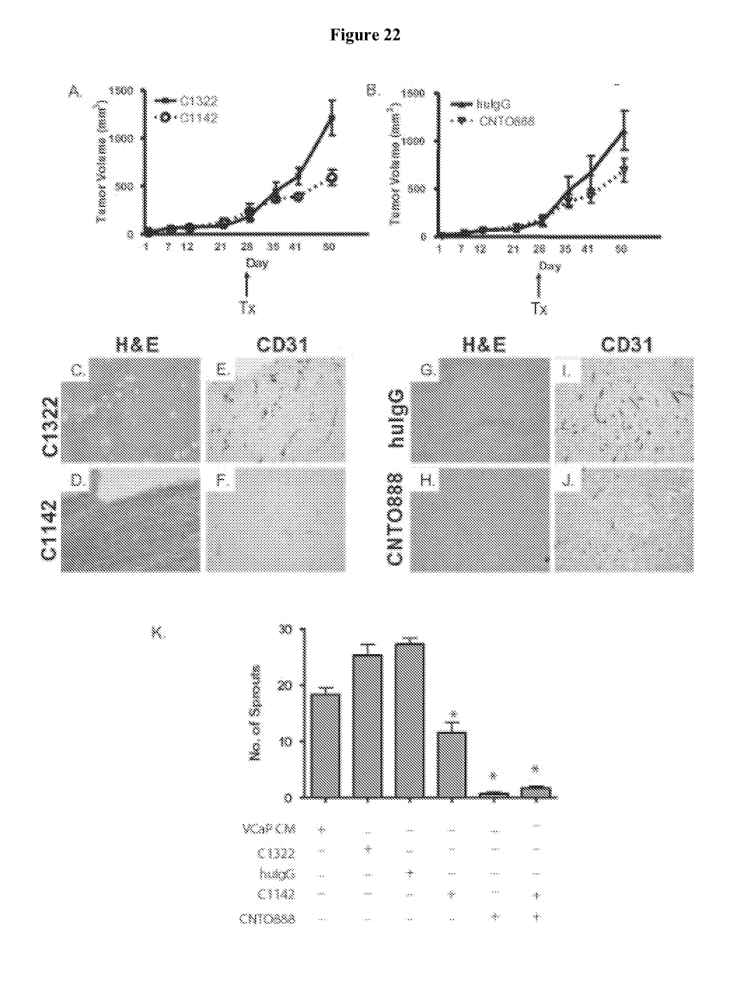

To determine the role of CCL2 in prostate cancer growth in vivo, VCaP xenografts were implanted in male SCID mice (n=5) and tumor growth was monitored by caliper measurement and calculation of tumor volume. Twenty-eight days post implantation, mice were divided into therapy groups: 1) huIgG control, 2) anti-human CCL2 antibody, CNTO888, 3) mouse control antibody C1322, and 4) anti-mouse CCL2/JE antibody C1142. Mice were treated with antibodies at 2 mg/Kg given twice weekly by intraperitoneal injection. Antibodies were delivered for 3 weeks and animals were sacrificed on Day 50. Tumor volume measurements revealed a reduction of 55.2% of tumor growth on Day 50 by administration of a neutralizing anti-mouse CCL2/JE antibody (C1142) compared to C1322 (FIG. 22a). Similarly, administration of a neutralizing anti-human CCL2 antibody (CNTO888) resulted in a 42.2% reduction in tumor volume on Day 50 compared to the huIgG control (FIG. 22b). These data indicate that inhibition of either tumor-derived human CCL2 or stromal mouse-derived CCL2/JE can significantly delay tumor growth of these tumors.

The xenograft tumors were collected for histological analysis and quantification of microvascular density. Sections were stained for CD31 (PECAM, a marker of vascular endothelium). Inhibition of CCL2 with either the anti-human CCL2 or the anti-mouse CCL2/JE neutralizing antibodies decreased the amount of angiogenesis as identified by a decrease in CD31 staining compared to isotype controls (FIGS. 22 e&f: anti-mouse, FIGS. 22 i&j: anti-human). To further elucidate the role of CCL2 inhibition on blood vessel formation, an in vitro tube formation assay was applied as previously described (Zhou et al., Int J Cancer 110 (2004) 800-806). Human dermal microvascular endothelial cells grown in VCaP conditioned media (CM) in MATRIGEL formed a capillary-like network of tubes (FIG. 22k). Administration of either CNTO888 or C1142 (30 μg/mL) to the VCaP conditioned media significantly reduced the number of capillary-like tubes that formed compared to the isotype control antibody treated cells (FIG. 22k).

CCL2 is known to promote monocyte/macrophage infiltration into tissue and the role of TAMs in prostate cancer biology has demonstrated a direct role in regulating tumor growth and angiogenesis. The macrophage infiltrate was assessed in the xenograft tumors by immunohistochemistry. Macrophages were identified by CD68 (lysosomal glycoprotein, a marker of monoctyes and macrophages) positive staining.

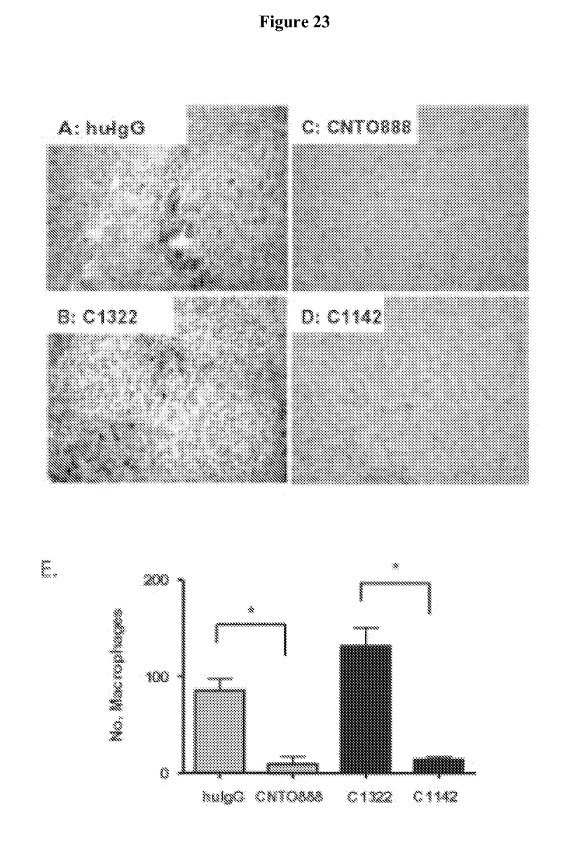

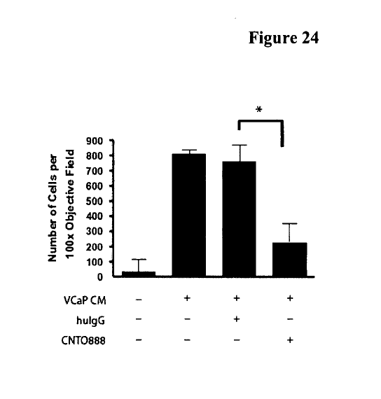

Inhibition of CCL2 attenuated monoctye/macrophage infiltration as evident by a lack of CD68 positive staining compared to the isotype controls (FIGS. 23 a&c: anti-human, FIGS. 23 b&d: anti-mouse). Macrophage infiltration was quantified by manual counting of CD68+ cells per 100× objective field and inhibition of CCL2 demonstrated a significant decrease in the number of CD68+ cells present within the VCaP xenograft (IgG: 85.33±12.10, CNTO888: 9.00±7.94, C1322: 131.00±19.08, C1142: 13.67±3.06, mean ±SD, p<0.0001) (FIG. 23e). Inhibition of macrophage migration using CNTO888 was confirmed in an in vitro migration assay (FIG. 24). VCaP conditioned media induced a significant increase in the number of migrating human U937 premonocytic cells (a pre-macrophage cell line). Pre-incubation of U937 cells with CNTO888 (30 μg/mL) significantly attenuated the migratory effect induced by the VCaP conditioned media (SFM: 33±69, VCAPCM: 805+28, huIgG: 756±136, CNTO888: 225±111; p<0.001).

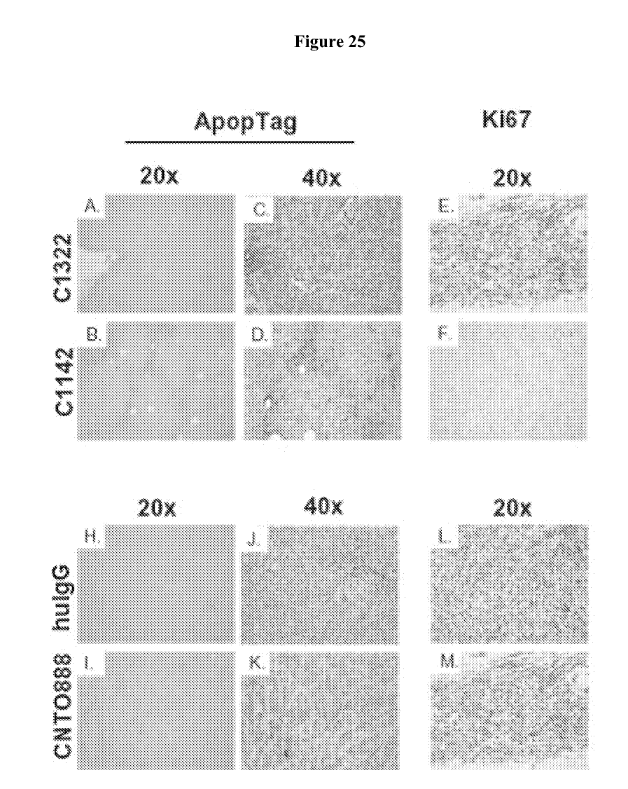

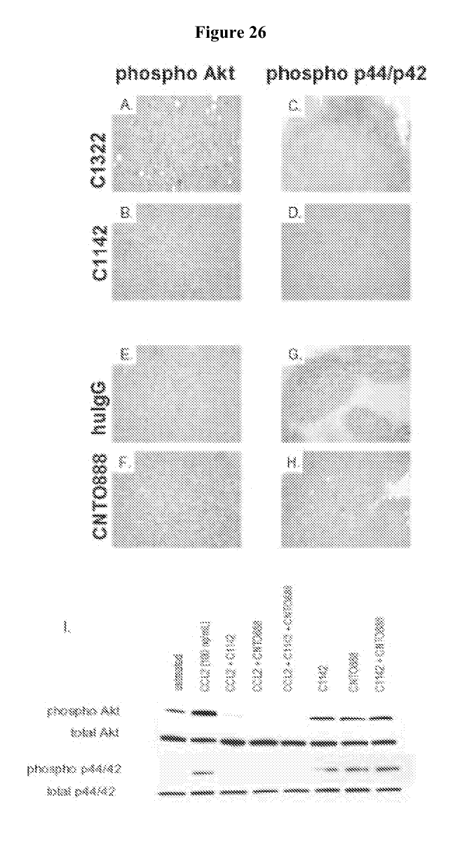

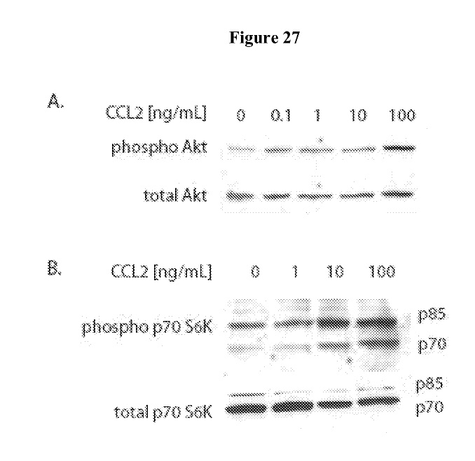

CCL2 and the presence of tumor associated macrophages have been shown to directly stimulate tumor cell proliferation (Lu et al., Prostate 66 (2006) 1311-1318; Loberg et al., Neoplasia 8 (2006) 578-586). The proliferative status of the VCaP xenografts was assesed by immunohistochemical staining for Ki67 (a marker of proliferation) and the effects on proliferation were compared with the effects on apoptosis by staining similar sections with an apoptosis stain (ApopTag) (FIG. 25). Inhibition of CCL2 resulted in a significant decrease in Ki67 staining and an induction of apoptosis. This was accompanied by a decrease in phosphorylated Akt and phosphorylated p44/p42 MAPK (FIG. 26a-h). VCaP cells stimulated with recombinant human CCL2 in vitro resulted in an increase in Akt phosphorylation and p44/p42 MAPK phosphorylation that was attenuated with the addition of CNTO888 and C1142 (FIG. 26i). VCaP cells were stimulated with increasing concentrations of human CCL2 in vitro and demonstrated dose dependent activation of Akt (FIG. 27a) and a dose dependent activation of p70 S6 kinase, a downstream target of Akt that is known to be important in cellular proliferation (FIG. 27b).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method of treating or preventing metastasis of a prostate cancer cell, comprising contacting said cancer cell with an agent that inhibits an activity of MCP-1.

2. The method of claim 1, wherein said metastasis is a bone metastasis.

3. The method of claim 1, wherein said agent is a small molecule that inhibits a biological activity of MCP-1.

4. The method of claim 1, wherein said agent is an antibody that binds to said MCP-1.

5. The method of claim 1, wherein said agent is an antisense or siRNA that inhibits the expression of said MCP-1.

6. The method of claim 1, wherein said agent is an MCP-1 TRAP.

7. The method of claim 1, wherein said agent is a combination of a known chemotherapy agent and an antibody that binds to said MCP-1.

8. The method of claim 7, wherein said known chemotherapy agent is docetaxel.

9. The method of claim 7, further comprising the step of following said contacting said cell with said combination of said antibody that binds to said MCP-1 and said known chemotherapy agent, contacting said cell with only said antibody that binds to said MCP-1.

10. The method of claim 1, wherein said cancer cell is in an organism.

11. The method of claim 10, wherein said organism is selected from the group consisting of a human and a non-human mammal.

12. A method for identifying prostate cancer likely to metastasize to the bone, comprising measuring the level of expression of MCP-1 in a prostate cancer tissue sample.

13. The method of claim 12, wherein an increase in MCP-1 relative to the level in a non-cancerous prostate tissue is indicative of the presence of prostate cancer in said tissue that is likely to metastasize.

14. The method of claim 12, further comprising the step of determining a treatment course of action based on said measuring.

15. A method of screening compounds, comprising
a) contacting a prostate cancer cell expressing MCP-1 with a test compound; and
b) determining the likelihood of said prostate cancer cell to metastasize based on the level of biological activity of MCP-1 in the presence of said test compound relative to the level in the absence of said test compound.

16. The method of claim 15, wherein said prostate cancer cell is in an organism.

17. The method of claim 16, wherein said organism is a non-human mammal.

18. The method of claim 15, wherein said test compound is selected from the group consisting of a small molecule, a known chemotherapy agent, an antibody, an siRNA, an MCP-1 TRAP, and an antisense nucleic acid.

19. The method of claim 18, wherein said test compound comprises a combination of a known chemotherapy agent and an antibody.

20. The method of claim 15, wherein said test compound decreases said biological activity of MCP-1.
